(19) United States
(12) Patent Application Publication
(10) Pub. No.: US 2016/0095823 A1
(43) Pub. Date: Apr. 7, 2016

(54) METHODS OF USING PROPOFOL DERIVATIVES FOR ANALGESIA

(71) Applicants: The University of Liverpool, Liverpool (GB); The University of Dundee, Dundee (GB); Medizinische Hochschule Hannover, Hannover (DE)

(72) Inventors: Martin Leuwer, Liverpool (GB); Gertrud Haeseler, Hannover (DE); Jeremy Lambert, Dundee (GB); Delia Belelli, Dundee (GB)

(73) Assignees: The University of Liverpool, Liverpool (GB); The University of Dundee, Dundee (GB); Medizinische Hochschule Hannover, Hannover (DE)

(21) Appl. No.: 14/951,003

(22) Filed: Nov. 24, 2015

Related U.S. Application Data
(63) Continuation of application No. 11/612,623, filed on Dec. 19, 2006.
(60) Provisional application No. 60/751,559, filed on Dec. 19, 2005.
(60) Provisional application No. 60/796,270, filed on Apr. 27, 2006.

(51) Int. Cl. A61K 31/055

(52) U.S. Cl. CPC A61K 31/055

(57) ABSTRACT

The present invention concerns compounds derived from the anaesthetic propofol that are useful as analgesics and methods of using the same. The compounds act as co-activators of strychnine-sensitive glycine receptors and may have greater activity at those glycine receptors than at GABA_{A}.

### CROSS-REFERENCE TO RELATED APPLICATIONS

This application is a continuation of U.S. patent application Ser. No. 11/612,623, filed Dec. 19, 2006, which includes a claim of priority under 35 U.S.C. §119(e) to U.S. provisional application Ser. No. 60/751,559, filed Dec. 19, 2005, and U.S. provisional application Ser. No. 60/796,270, filed Apr. 27, 2006.

### FIELD OF THE INVENTION

The present invention relates to the use of phenol derivatives as analgesics.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Effective safe pain control is regarded worldwide as a high priority clinical need. However the majority of developments in this field have failed to deliver high efficacy products free of undesirable side effects and safety issues. The opiates probably remain as the most effective treatment available and the ultimate goal is to deliver a pain control agent with the efficacy of the opiates but without the sedation, dependence, gastric damage and general tolerability problems.

It has been postulated that phenol derivatives may have a number of neuromodulatory effects. However the only phenol derivative in widespread clinical use is the anaesthetic propofol (2,6-di-isopropylphenol).

Key features of anaesthesia are loss of consciousness, immobility in the presence of painful stimuli and absence of recall. Anaesthetics, such as propofol, are understood to mediate their anesthetic effect by activating γ-aminobutyric acid (GABA_{A}) receptors in the Central Nervous System (CNS).

In contrast, analgesia is defined as the absence of pain. Among other peripheral and/or central nervous mechanisms, analgesia can arise as a result of enhanced inhibitory synaptic transmission within the dorsal horn of the spinal chord. It is understood that inhibitory postsynaptic transmission in the spinal chord involves mainly glycine receptors. Accordingly the glycine receptor family represents a target site for therapeutic agents aiming at inhibiting pain.

Both, GABA_{A} and glycine receptors belong to the ligand-gated ion channel superfamily. They have a common structure in which five subunits form an ion channel. α and β subunits assemble into a pentameric receptor with a proposed in vivo stochiometry of 3α: 2β. Glycine receptors, like GABA_{A} receptors, inhibit neuronal firing by opening chloride channels following agonist binding. Glycine receptors are mainly found in lower areas of the central nervous system and are involved in the control of motor rhythm generation, the coordination of spinal nociceptive reflex responses and the processing of sensory signals.

There exists a need to develop new and improved analgesics. Despite that fact that glycine receptors represent a good target for identifying such analgesics, there are no existing analgesics that target these receptors. The inventors therefore decided to address this issue and exploited their knowledge of the pathophysiological mechanisms underlying anaesthesia and analgesia with a view to identifying new and improved drugs for controlling pain.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1: illustrates the chemical structures of the phenol derivatives tested in Example 1 from top to bottom: the compounds with the methyl groups in ortho position to the phenolic hydroxyl group; the compound with a single methyl group in meso position and its halogenated analogue; and the compound with two methyl groups in meso position and its halogenated analogue.

FIG. 2: FIG. 2A: Representative current traces elicited by 2 s application of 3,5-dimethylphenol or 2,6-dimethylphenol with respect to the current elicited by 1000 μM glycine in the same experiment (upper trace). Tracings were obtained from one HEK 293 cell each expressing either α₁ homomeric or α₁β glycine receptors. FIG. 2B: Normalized Cl⁻ currents activated in the absence of glycine via α₁ homomeric (triangles) or α₁β (circles) glycine receptors (mean±SD; n=3 each), plotted against the concentration of 3,5 dimethylphenol (upper diagram) or 2,6 dimethylphenol (lower diagram) on a logarithmic scale. Currents were normalized either to maximum value achieved by high concentrations (3000 μM) of the compound (filled symbols) or to maximum value achieved by 1000 μM glycine (empty symbols). Solid lines are Hill fits to the data with the indicated parameters. The concentration-response plots were almost superimposable for α₁ homomeric and α₁β glycine receptors, and no difference between the ortho- and meso-methylated compound could be detected; all as discussed in Example 1.

FIG. 3: Normalized Cl⁻ currents activated by glycine via α₁ homomeric (triangles) or α₁β (circles) glycine receptors (mean±SD; n=3 each), plotted against the concentration of glycine as referred to in Example 1. Solid lines are Hill fits to the data with the indicated parameters.

FIG. 4: Representative current traces elicited by 2 s co-application of 10 μM glycine and (from left to right) 2-methylphenol, 3-methylphenol, and 3-methyl-4-chlorophenol with respect to the current elicited by 1000 μM glycine in the respective control experiment (upper trace) in α₁β heteromeric receptors as described in Example 1. All compounds increased the amplitude of the response evoked by 10 μM glycine. In the halogenated compound (right row of traces) this effect was observed in the low μM concentration range.

FIG. 5: Representative current traces elicited by 2 s co-application of 10 μM glycine and (from left to right) 2,6 dimethylphenol, 3,5 dimethylphenol, and 3,5 dimethyl-4-chlorophenol with respect to the current elicited by 1000 μM glycine in the respective control experiment (upper trace) in α₁β heteromeric receptors as described in Example 1. The halogenated compound (right row of traces) showed co-activating effects in the low μM concentration range.

FIG. 6: Representative current traces elicited via α₁ homomeric receptors by 2 s co-application of 10 μM glycine with either 3,5 dimethylphenol (upper row of traces), 3 methylphenol (lower row of traces) or their respective halogenated analogue (right row of traces) with respect to the current elicited by 1000 μM glycine (upper trace) as described in Example 1. The effect elicited by 10 μM glycine is higher in α₁ homomeric receptors than in α₁β heteromeric receptors (compare with tracings in FIGS. 3 and 4). Co-activating effects of phenol derivatives in α₁ homomeric receptors are seen in a similar concentration range compared to α₁β heteromeric receptors.

FIG. 7: Potentiation (%) of the current elicited by 10 μM glycine (mean±SD of 5-6 independent experiments) by each compound in α₁β heteromeric receptors, plotted against the concentration applied on a logarithmic scale as described in Example 1. Solid lines are Hill fits to the data with the parameters indicated in Table 1. The concentrations required for a half-maximum co-activating response were significantly smaller in the halogenated compounds compared with their non-halogenated structural analogues (p<0.0001). No significant differences between the compounds were detected with respect to the degree of maximum potentiation. Only the potentiating effect seen with 3 methylphenol was higher than with 2 methylphenol (p=0.04), which, however, might be a consequence of the lower response to glycine 10 μM in the experiments with 3 methylphenol with respect to the experiments with 2 methylphenol.

FIG. 8: Inhibitory effects induced by 3 methyl-4-chlorophenol (left row of traces) and 3,5 dimethyl-4-chlorophenol (right row of traces) at concentrations≧600 and 300 μM, respectively, as revealed by a reduction in the peak current amplitude during co-application with glycine 1000 μM, a concentration-dependent acceleration of the current decay during application followed by channel reopening at the end of the application as described in Example 1.

FIG. 9: Representative current traces, as discussed in Example 2, showing co-activation of the current response to 10 μM glycine when 4-chloropropofol was co-applied with 10 μM glycine (3rd, 4th, 5th and 6th current trace from top). The first trace shows current elicited by a supramaximal glycine concentration (1000 μM) FIG. 10: Concentration-dependence of the coactivation of the current response to 10 μM glycine by 4-chloropropofol (mean/SD, n=6) as discussed in Example 2.

FIG. 11: Concentration-dependence of the coactivation of the current response to 10 μM glycine by 4-chloropropofol (mean/SD, n=5) as discussed in Example 2. The solid line is a Hill fit to the data with the indicated parameters.

FIG. 12: Representative current traces, as discussed in Example 2, elicited by 4-chloropropofol in the absence of the natural agonist glycine (2nd, 3rd and 4th trace from top) with respect to the current elicited by a supramaximal glycine concentration (1000 μM), top.

FIG. 13: Normalized Cl⁻ currents (mean±SD, n=4) activated by 4-chloropropofol normalized to its own maximum response (empty symbols) or the maximum response elicited by 1000 μM glycine (filled symbols) plotted against the concentration of glycine as referred to in Example 2. Solid line is Hill fit to the data with the indicated parameters.

FIG. 14: Representative current traces, as discussed in Example 2, showing co-activation of the current response to 10 μM glycine when 4-bromopropofol was co-applied with 10 μM glycine (3rd, 4th, 5th and 6th current trace from top). The first trace shows current elicited by a supramaximal glycine concentration (1000 μM) FIG. 15: Concentration-dependence of the coactivation of the current response to 10 μM glycine by 4-bromopropofol (mean/SD, n=5) as discussed in Example 2. The solid line is a Hill fit to the data with the indicated parameters.

FIG. 16: Representative current traces, as discussed in Example 2, elicited by 4-bromopropofol in the absence of the natural agonist glycine (2nd, 3rd and 4th trace from top) with respect to the current elicited by a supramaximal glycine concentration (1000 μM), top.

FIG. 17: Normalized Cl⁻ currents (mean±SD, n=4) activated by 4-bromopropofol normalized to its own maximum response (empty symbols) or the maximum response elicited by 1000 μM glycine (filled symbols) plotted against the concentration of glycine as referred to in Example 2. Solid line is Hill fit to the data with the indicated parameters.

FIG. 18: Representative current traces, as discussed in Example 2, showing co-activation of the current response to 10 μM glycine when 4-iodopropofol was co-applied with 10 μM glycine (3rd, 4th, 5th and 6th current trace from top). The first trace shows current elicited by a supramaximal glycine concentration (1000 μM) FIG. 19: Concentration-dependence of the coactivation of the current response to 10 μM glycine by 4-iodopropofol (mean/SD, n=4) as discussed in Example 2. The solid line is a Hill fit to the data with the indicated parameters.

FIG. 20: Representative current traces, as discussed in Example 2, elicited by 4-iodopropofol in the absence of the natural agonist glycine (2nd, 3rd and 4th trace from top) with respect to the current elicited by a supramaximal glycine concentration (1000 μM), top.

FIG. 21: Normalized Cl⁻ currents (mean±SD, n=4) activated by 4-iodopropofol normalized to its own maximum response (empty symbols) or the maximum response elicited by 1000 μM glycine (filled symbols) plotted against the concentration of glycine as referred to in Example 2. Solid line is Hill fit to the data with the indicated parameters.

FIG. 22: FIG. 22A illustrates tadpoles used according to Example 3, including Stage 37/8 embryos (FIG. 22Ai), inter-myotomal clefts (FIG. 22Aii), and positioning of electrodes (FIG. 22Aiii); FIG. 22B is an illustrative trace for fictive swimming recorded at rostral, contra and caudal positions, including whole recordings (FIG. 22Bi) and excerpts therein (FIG. 22Bii).

FIG. 23: illustrates the effect of 4 chloropropofol on fictive swimming in tadpoles as discussed in Example 3 at 3.3.1, including whole recordings (FIG. 23Ai) and excerpts therein (FIG. 23Aii) and pooled cycle data (FIG. 23B).

FIG. 24: represents tabulated data discussed in Example 3 at 3.3.1, including cycle period (FIG. 24A) and episode duration (FIG. 24B).

FIG. 25: illustrates the effect of 4 chloropropofol and bicuculline methiodide on ventral root activity in tadpoles as discussed in Example 3 at 3.3.2, including bicuculline (FIG. 25A) and further with 4-chloropropofol (FIG. 25B).

FIG. 26: represents tabulated data discussed in Example 3 at 3.3.2 including cycle period (FIG. 26A) and episode duration (FIG. 26B).

FIG. 27: illustrates the effect of 4 chloropropofol and strychnine on ventral root activity in tadpoles as discussed in Example 3 at 3.3.3, including ventral root activity (FIG. 27Ai), with 4-chloropropofol (FIG. 27Aii), and pooled cycle period (FIG. 27B).

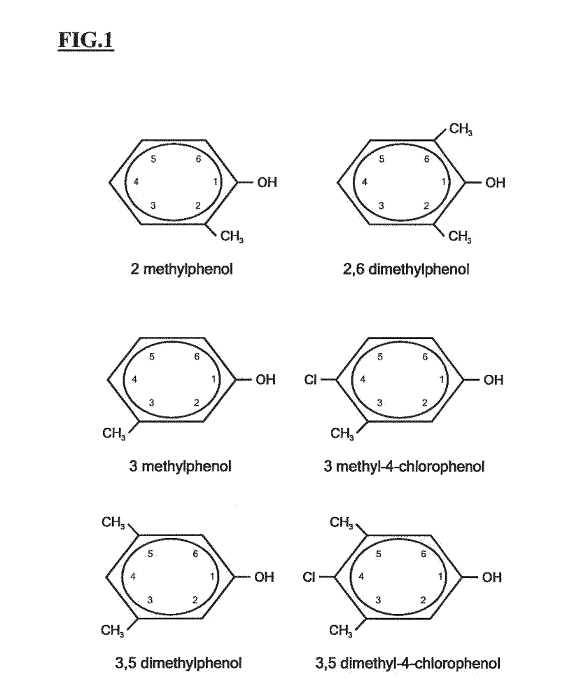

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

According to a first aspect of the present invention there is provided a use of a compound of general formula I:

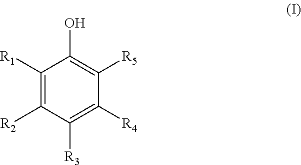

wherein R₃ is a halogen, amine or amide; R₁, R₂, R₄ and R₅ are independently H, or an alkyl comprising between 1 and 13 carbon atoms; and salts thereof; in the manufacture of a medicament for the treatment of pain. In an embodiment, at least two of R₁, R₂, R₄ and R₅ are an alkyl comprising between 2 and 13 carbon atoms.

According to second aspect of the present invention there is provided a method of treating pain in a subject in need of such treatment comprising administering to said subject a therapeutically effective amount of a compound of general formula I.

According to third aspect of the invention there is provided a compound as defined by the first aspect of the invention for use as a medicament.

The inventors recognized that a loss of inhibitory synaptic transmission within the dorsal horn of the spinal cord plays a key role in the development of chronic pain following inflammation or nerve injury. Furthermore they recognized that inhibitory postsynaptic transmission in the spinal cord involves mainly glycine. This led them to realise that the strychnine-sensitive glycine receptor family represents a target site for therapeutic agents aiming at inhibiting pain sensitization. This realization was based upon work conducted by Ahmadi et al. (Nature Neuroscience (2001) Vol. 5 No. 1 p34-40).

The inventors therefore endeavoured to develop analgesics that specifically target glycine receptors and to their surprise, they discovered that compounds according to the first aspect of the invention act as co-activators of glycine receptors. Compounds according to the first aspect of the invention may be ligands for strychnine-sensitive glycine receptors. The compounds may bind to strychnine-sensitive glycine receptors with more specificity than GABA receptor types in the CNS.

The inventors proceeded by making derivatives of the anaesthetic propofol and were surprised to find that compounds of general formula I are extremely potent positive allosteric modulators of strychnine sensitive glycine receptors.

The inventors used propofol as a starting point because they recognized that the glycine receptor α1 subunit shares primary sequence homology with transmembrane segments of α, β and γ subunits of the GABA_{A} receptor in a region of the subunit that harbours amino acid residues crucial for the binding of alcohols, volatile anaesthetics and propofol. Furthermore they recognized that propofol had been reported to activate glycine receptors at 10-fold higher concentrations than its EC₅₀ at GABA_{A} receptors. As this means that the desired glycine activating effect of propofol would only be available at concentrations that in clinical practice would induce deep coma, they decided to generate other derivatives of phenol to investigate whether or not such derivatives would exhibit improved agonist activity at glycine receptors and thereby be useful as analgesics.

The inventors were surprised to find that compounds of general formula I were extremely potent at co-activating glycine receptors and were therefore useful as analgesics. While not wishing to be bound by any particular theory, they believe that the introduction of R₃ at the para position of phenol and the introduction of the specified alkyl or alkylene-containing groups results in phenol derivatives with EC₅₀ values for co-activation of Glycine receptors that were significantly (by up to three orders of magnitude) lower than the most potent compounds known to the art. The compounds described in the Examples showed half-maximum potentiating effects in the low μM range. This represents more than 20-fold lower concentrations than for propofol.

The compounds may have selectivity for strychnine-sensitive glycine receptors over GABA_{A} receptors. The compounds may have an EC₅₀ for co-activating glycine receptors at a lower concentration than its EC₅₀ at GABA_{A} receptors. In an embodiment, the compounds have an EC₅₀ for co-activating glycine receptors that is 10-fold lower than its EC₅₀ at GABA_{A} receptors. The compounds may have an EC₅₀ for co-activating glycine receptors that is at least 100-fold lower than its EC₅₀ at GABA_{A} receptors.

The compounds may also have an EC₅₀ for co-activating glycine receptors that is lower than that of propofol. For instance the compound may have an EC₅₀ for co-activating glycine receptors that is at least 10-fold lower or 100-fold lower than that of propofol. Certain compounds of the invention (e.g. 2,6 di-isopropyl-4-chlorophenol) have an EC₅₀ for co-activating glycine receptors that is 1000-fold lower than that of propofol (when measured on glycine receptors heterologously expressed in HEK293 cells).

Suitable methods for measuring EC₅₀ values for co-activating glycine receptors are disclosed in Example 1.

The efficacy of the compounds is all the more surprising when the neurophysiology modulating anaesthesia in the CNS and analgesia in the PNS is considered. While not wishing to be bound by any particular theory, the inventors believe that compounds according to general formula I act as positive allosteric modulators at strychnine-sensitive glycine receptors. These receptors are chloride channels that stabilise membrane potential by hyperpolarisation and constitute the predominant inhibitory principle at the spinal cord level. In contrast, the closely related GABA_{A} receptor constitutes the predominant inhibitory principle in the CNS. A GABA_{A} agonistic drug will, therefore, lead to an alteration or a loss of consciousness, whereas a compound according to the invention will ideally block pain at the peripheral level at concentrations that will not affect consciousness. The inventors believe compounds according to the invention have efficacy because they act as positive allosteric modulators at strychnine-sensitive glycine receptors and thereby block centripetal nerve signals at the dorsal root ganglionic level but have minimal or no effects at central GABA_{A} receptors. It therefore follows, that a skilled person would choose an analgesic that was a glycine receptor agonist and which would have no GABA_{A} agonistic effect at all. Consequently, propofol, which is the most potent GABA_{A} agonist known, would be regarded by a skilled person as the least suitable compound to serve as a platform for developing analgesics. The inventors therefore believe that there was a technical prejudice against investigating the analgesic properties of propofol derivatives. Thus, the extra-extra-ordinary increase in glycine agonistic potency which the inventors found with compounds according to the invention was not only surprising but would have been considered unlikely by the skilled artisan. In theory, any other phenol derivative with a lesser potency at the GABA_{A} receptor level should, according to the state of the art, have been considered to be a more promising candidate.

In various embodiments of the invention, R₃ is a halogen. The halogen may be Fluorine, Chlorine, Iodine or Bromine. The results presented in Example 2 demonstrate that unexpectedly good efficacy is exhibited by compounds falling within the scope of the present invention that are substituted at the 4-position with any desirable halogen, particularly chlorine, bromine or iodine. These results support the proposed physiological mechanism, i.e. positive modulation of the effect of a submaximal concentration of the natural transmitter glycine. Moreover, the 4-chloro-, 4-bromo- and 4-iodo-derivatives of 2,6-di-isopropylphenol tested in Example 2 all exhibited very low ECM) values in the single-digit nanomolar range. R₁, R₂, R₄ or R₅ may each separately comprise a methylene group and, in certain embodiments, may be a methyl group (i.e. a protonated methylene group). When the alkyl group comprises a methylene group, two of R₁, R₂, R₄ and R₅ may be H and the other two may comprise a methylene group. In certain embodiments, two of R₁, R₂, R₄ and R₅ are methyl groups. Methylated phenol derivatives for use according to the invention may include 3,5 dimethyl-4-chlorophenol or 2,6 dimethyl-4-chlorophenol.

In certain embodiments, two of R₁, R₂, R₄ and R₅ may be H and the other two may be an alkyl comprising between 2 and 13, and, in certain embodiments, between 3 and 6 carbon atoms. The two alkyl groups may be both in the ortho or meso positions, respectively. Accordingly, in certain embodiments, R₁ and R₅ may be alkyl groups and R₂ and R₄ may be hydrogen or alternatively R₂ and R₄ may be alkyl groups and R₁ and R₅ may be hydrogen.

R₁, R₂, R₄ and R₅ may each separately be a hydrogen atom or a straight or branched substituted or unsubstituted alkyl group, such as an ethyl group, n-propyl, iso-propyl group, n-butyl group or an iso-butyl group. In certain embodiments, two of R₁, R₂, R₄ and R₅ are iso-propyl groups, preferably with the remaining two of R₁, R₂, R₄ and R₅ being hydrogen atoms. Examples of compounds for use according to the invention include: 2,6-di-isopropyl-4-chlorophenol, 2,6-di-isopropyl-4-iodophenol, 2,6-di-isopropyl-4-fluorophenol, 2,6-di-isopropyl-4-bromophenol, 3,5-di-isopropyl-4-chlorophenol, 3,5-di-isopropyl-4-iodophenol, 3,5-di-isopropyl-4-flurophenol and 3,5-di-isopropyl-4-bromo-phenol.

2,6-di-isopropyl-4-chlorophenol, 2,6-di-isopropyl-4-bromophenol, and 2,6-di-isopropyl-4-iodophenol are illustrative of compounds that are suitable for use according to the invention. The inventors have demonstrated (see the examples) that each of these compounds surprisingly enhances the function of glycine receptors heterologously expressed in HEK293 cells at 1000-fold lower concentrations than the parent compound propofol. A skilled person will appreciate that this makes these compounds particularly useful as an analgesic as described herein.

Compounds according to the invention and medicaments containing such compounds may be used as analgesics in a number of circumstances.

The compounds are particularly useful for targeting chronic pain states (e.g. neuropathic and/or post-inflammatory chronic pain) that, so far, have been notoriously difficult to treat. The compounds are particularly useful for treating chronic neuropathic pain which is hard to treat with conventional drugs such as NSAIDs, opiate derivatives, etc. The compounds are also useful for treating acute pain (e.g. following injury).

The compounds of the invention are also beneficial because they avoid all the familiar side effects of local anaesthetics and analgesics as well as NSAIDs and opioids if used as a monotherapy while, at the same time, allowing a vast variety of combined treatment strategies aiming at additive or supra-additive effects.

Examples of specific conditions in which pain may be modulated include chronic lower back pain, arthritis, cancer pain, trigeminal neuralgia, stroke and neuropathic pain.

The compounds may be used to treat existing pain but may also be used when prophylactic treatment is considered medically necessary, for instance, in advance of elective surgery.

The compounds may be used as an analgesic in the form of a monotherapy (i.e., use of the compound alone) or alternatively the compounds may be given in combination with other treatments that also reduce pain. Combination therapy may involve the use of the compounds with analgesics that modulate pain by a pain processing pathway that is different to the pathway(s) modulated by compounds of general formula. Such analgesics may include morphine, paracetamol, and NSAIDs. The compounds may also be usefully combined with local anaesthetics (e.g. lignocaine) that only indirectly interact with glycine receptors.

The medicaments of the invention may comprise a compound of general formula I and a pharmaceutically acceptable vehicle. It will be appreciated that the vehicle should be one which is well tolerated by the subject to whom it is given and enables delivery of the compounds to the affected area.

The medicaments of the invention may take a number of different forms depending, in particular, on the manner in which the compound is to be used. Thus, for example, the medicament may comprise a compound in the form of a salt of the phenol derivative (e.g. a sodium salt). Such salts may be manufactured in a powder form and incorporated in a tablet, capsule, liquid, ointment, cream, gel, hydrogel, aerosol, spray, micelle, transdermal patch, liposome or any other suitable form that may be administered to a person or animal.

Alternatively the phenol derivative according to the invention may be dissolved in a suitable solvent to form a liquid medicament. The solvent may be aqueous (e.g. PBS or distilled water). Alternatively the solvent may be an alcohol such as ethanol or a mixture of such a solvent with an aqueous solvent.

The medicament may be used for topical or local treatment. Such medicaments may be formulated as a liquid for application to an effected site. Alternatively the liquid may be formulated for administration by injection or as an aerosol.

The compound may also be incorporated within a slow or delayed release device. Such devices may, for example, be inserted on or under the skin and the compound may be released over weeks or even months. Such a device may be particularly useful for patients with long-term chronic pain (e.g. a patient with arthritis). The devices may be particularly advantageous when a compound is used which would normally require frequent administration.

It will be appreciated that the amount of a compound required is determined by biological activity and bioavailability which in turn depends on the mode of administration, the physicochemical properties of the compound employed and whether the compound is being used as a monotherapy or in a combined therapy. The frequency of administration will also be influenced by the abovementioned factors and particularly the half-life of the compound within the subject being treated.

Optimal dosages to be administered may be determined by those skilled in the art, and will vary with the particular compound in use, the strength of the preparation, the mode of administration, and the extent of the pain requiring relief. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials, etc.), may be used to establish specific formulations of compositions and precise therapeutic regimes (such as daily doses of the compounds and the frequency of administration), and can be carried out by those of skill without undue experimentation.

Generally, a dose should be given that is effective for delivering a compound at the target site such that the tissue concentration is around the EC₅₀ of the compound used.

Daily doses may be given as a single administration (e.g. as a single daily injection). Alternatively, the compound used may require administration twice or more times during a day. As an example, 2,6-di-isopropyl-4-chlorophenol, for treating chronic lower back pain may be administered as two (or more depending upon the severity of the pain) daily doses of an injectable solution or an ointment. A patient receiving treatment may take a first dose upon waking and then a second dose in the evening (if on a two dose regime) or at 3 or 4 hourly intervals thereafter. Alternatively, a slow release device may be used to provide optimal doses to a patient without the need to administer repeated doses.

This invention further provides a pharmaceutical composition comprising a therapeutically effective amount of the compound of the invention and a pharmaceutically acceptable vehicle. In one embodiment, the amount of a salt of a phenol derivative (e.g. 2,6-di-isopropyl-4-chlorophenol) is an amount from about 10 μg/kg Body Weight to 10 mg/kg Body Weight in each dose unit for enteral (oral, rectal) administration. In another embodiment, the amount is from about 1 μg/kg Body Weight to 1 mg/kg Body Weight in each dose unit for parenteral (intravenous/intrathecal or epidural) administration.

In a further embodiment, the vehicle is a liquid and the composition is a solution. Useful liquid solutions for parenteral administration may comprise between 0.001 and 1% by weight of the phenols of formula I. In another embodiment, the vehicle is a solid and the composition is a tablet. In a further embodiment, the vehicle is a gel and the composition is for topical application.

In the subject invention a “therapeutically effective amount” is any amount of a compound, medicament or composition which, when administered to a subject suffering from a painful condition against which the compounds are effective, causes reduction, remission, or regression of the pain.

A “subject” is a vertebrate, mammal, domestic animal or human being.

In the practice of this invention the “pharmaceutically acceptable vehicle” is any physiological vehicle known to those of ordinary skill in the art useful in formulating pharmaceutical compositions. In one embodiment, the pharmaceutical vehicle may be a liquid and the pharmaceutical composition would be in the form of a solution. In another embodiment, the pharmaceutically acceptable vehicle is a solid and the composition is in the form of a powder or tablet. In a further embodiment, the pharmaceutical vehicle is a gel and the composition is in the form of a suppository or cream. In a further embodiment the compound or composition may be formulated as a part of a pharmaceutically acceptable transdermal patch.

A solid vehicle can include one or more substances which may also act as lubricants, solubilizers, suspending agents, fillers, glidants, compression aids, binders or tablet-disintegrating agents; it can also be an encapsulating material. In powders, the vehicle is a finely divided solid which is in admixture with the finely divided active ingredient. In tablets, the active ingredient is mixed with a vehicle having the necessary compression properties in suitable proportions and compacted in the shape and size desired. The powders and tablets preferably contain up to 99% of the active ingredient. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles are used in preparing solutions, suspensions, emulsions and the like. The phenol derivative can be dissolved or suspended in a pharmaceutically acceptable liquid vehicle such as water, ethanol, an organic solvent or mixtures thereof or pharmaceutically acceptable oils or fats.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium.

The invention also provides a method of screening a compound for efficacy as an analgesic, the method comprising applying a test compound to a tadpole and monitoring the effect of the compound on GABA neurotransmission mediated behavior and monitoring the effect of the compound on glycine neurotransmission mediated behavior wherein a compound that exclusively or mostly induces behavior characteristic of glycine neurotransmission is a putative analgesic.

Various embodiments of the screening method are disclosed in Example 3.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Experiments were conducted to investigate the effect of a number of phenol derivatives on Glycine receptor activation and chloride currents. A skilled person will appreciate that compounds which activate such receptors will be suitable for use as analgesics.

### 1.1 Methods

### 1.1.1 Cell Culture, Transfection

Rat α₁ and α₁β glycine receptor subunits were transiently transfected into transformed human embryonic kidney cells (HEK 293). α1 glycine receptor subunits efficiently form homomeric receptors in heterologous expression systems. β subunits do not form homomeric receptors but affect the function of heteromeric receptors, i.e. decreasing the sensitivity to the agonistic effect of glycine and to the blocking effects of picrotoxin analogues. When co-transfecting the glycine receptor α and β subunits, their respective cDNAs were combined in a ratio of 1:10, since expression of the β polypeptide is less efficient than that of the α subunits. Reduced sensitivity to 1000 μM picrotoxin in α₁β heteromeric receptors was used as an assay of the efficacy of β subunit expression. Cells were cultured in Dulbecco's modified Eagle's medium (DMEM, Biochrom, Berlin, Germany), supplemented with 10% fetal calf serum (FCS, Biochrom, Berlin, Germany), 100 U ml⁻¹ penicillin and 100 μg ml⁻¹ streptomycin at 37° C. in a 5% CO₂/air incubator. For transfection, cells were suspended in a buffer containing 50 mM K₂HPO₄ and 20 mM K-acetate, pH 7.35. For co-transfection of rat α₁ and β glycine receptor subunits, the corresponding cDNA, each subcloned in the pCIS2 expression vector (Invitrogen, San Diego, USA) was added to the suspension. To visualize transfected cells, they were co-transfected with cDNA of green fluorescent protein (GFP 10 μg ml⁻¹). For transfection, we used an electroporation device by EquiBio (Kent, UK). Transfected cells were replated on glass-coverslips and incubated 15-24 h before recording.

### 1.1.2 Chemicals & Solutions

All chemicals were from Sigma Chemicals (Deisenhofen, Germany), unless otherwise noted.

The phenol derivatives under investigation were prepared as 1 M stock solution in ethanol, light-protected and stored in glass vessels at −20° C. Concentrations were calculated from the amount injected into the glass vials. Drug-containing vials were vigorously vortexed for 60 min. Glycine and picrotoxin were dissolved directly in bath solution.

Patch electrodes contained [mM] KCl 140, MgCl₂ 2, EGTA 11, HEPES 10, glucose 10; the bath solution contained [mM] NaCl 162, KCl 5.3, NaHPO₄ 0.6, KH₂PO₄ 0.22, HEPES 15, glucose 5.6.

### 1.1.3 Experimental Set-Up

Standard whole-cell experiments (Hamill et al., (1981) Pflügers Arch., 391, 85-100.) were performed at −30 mV membrane potential. A tight electrical seal of several GΩ formed between the cell membrane and a patch-clamp electrode allows inward currents due to agonist-induced channel activation to resolve in the pA range. Electrical resistance of the pipettes was around 5 MΩ, corresponding to a total access resistance in the whole-cell configuration of about 10 MΩ. An ultra-fast liquid filament switch technique (Franke et al., (1987) Neurosci. Lett., 77, 199-204) was used for the application of the agonist in pulses of 2 s duration. The agonist and/or the drug under investigation were applied to the cells via a smooth liquid filament achieved with a single outflow (glass tubing 0.15 mm inner diameter) connected to a piezo crystal. The cells were placed at the interface between this filament and the continuously flowing background solution. When a voltage pulse was applied to the piezo, the tube was moved up and down onto or away from the cell under investigation. Correct positioning of the cell in respect to the liquid filament was ensured applying a saturating (1000 μM) glycine pulse before and after each test experiment. Care was taken to ensure that the amplitude and the shape of the glycine-activated current had stabilized before proceeding with the experiment. Test solution and glycine (1000 μM) were applied via the same glass-polytetrafluoroethylen perfusion system, but from separate reservoirs. The contents of these reservoirs were mixed at a junction immediately before entering the superfusion chamber.

Drugs were applied either alone, in order to determine their direct agonistic effects, in combination with a sub-saturating glycine concentration (10 μM), in order to determine their co-activating effects, or together with a saturating (1000 μM) concentration of glycine in order to detect open channel block. A new cell was used for each drug and each protocol, at least three different experiments were performed for each setting. The amount of the diluent ethanol corresponding to the highest drug concentration used was 34 000 μM. We have previously shown that the ethanol itself has no effect at this concentration—neither on glycine receptor co-activation, nor on direct activation.

### 1.1.4 Current Recording and Analysis

For data acquisition and further analysis the inventors used the Axopatch 200B amplifier in combination with pClamp6 software (Axon Instruments, Union City, Calif., USA). Currents were filtered at 2 kHz. Fitting procedures were performed using a non-linear least-squares Marquardt-Levenberg algorithm. Details are provided in the appropriate figure legends or in the results section.

The maximum current response induced by a compound acting directly as an agonist was expressed as percentage of the maximum response to 1000 μM glycine in the absence of drug immediately following the respective test experiment. The co-activating effect was expressed as percentage of the current elicited by 10 μM glycine according to E (%)=100 [(I−I₀)/I₀], where I₀ is the current response to 10 μM glycine. Activated or co-activated currents were normalized to their own maximum response. For the non-halogenated compounds, the dose-response curves did not always reach a plateau response, because phenol derivatives in concentrations larger than 3000 μM lead to a decline in seal resistance and thus, did not yield reliable results. In these cases, the maximum response was the response at the highest concentration of the test compound for which a reliable response could be recorded. The dose-response-curves were fitted according to (Iₙₒᵣₘ=[1+(EC₅₀/[C])^{nH}]⁻¹), where Iₙₒᵣₘ is the current induced either directly by the respective concentration [C] of the agonist, or co-activated (I−I₀) by the agonist-glycine (10 μM) mixture, normalized to the maximum inward current or maximum co-activated current (Iₘₐₓ−I₀), EC₅₀ is the concentration required to evoke a response amounting to 50% of their own maximal response and n_{H} is the Hill coefficient.

### 1.1.5 Statistics

Only results obtained with α₁β receptors were enrolled in the statistical tests. As a consequence of the higher glycine sensitivity in α₁ homomeric receptors, a maximum co-activating response (with respect to the effect of 1000 μM glycine) might occasionally be observed at low drug concentrations leading to an underestimation of the EC₅₀ values derived from Hill-fits in α₁ homomeric receptors. Statistical analysis was performed in order to reveal differences in the maximum effect on the one hand and in the concentrations required to achieve half-maximum effect (EC₅₀) on the other between halogenated and non-halogenated analogues, between compounds with one vs. two methyl groups, or between compounds with ortho- or meso-position of the methyl groups with respect to the phenolic hydroxyl group, respectively. Curve fitting and parameter estimation of the Hill curves were performed using the program “PROC NLMIXED” of SAS Release 8.02. In this model, the “experiment” is treated as the subject variable and the parameter values (EC₅₀ and n_{H}) are treated as normally distributed random factors. The mean differences of these parameters between two substances were entered into the common model as fixed shift parameters ΔEC₅₀ and Δn_{H}, activated for all data of the 2^{nd} data set. The corresponding (asymptotic) t-value was used to test the null hypothesis of no parameter difference against the two-sided alternative. The null hypothesis was rejected at p<0.05. All data are depicted as means±SD.

A two sample t-test was applied to analyze significance of differences in the maximum potentiating effect between halogenated and non-halogenated, mono- and bimethylated or ortho-vs. meso-methylated structural analogues.

### 1.2 Results

A total of 94 cells were included in the study. Expression of rat α₁ homomeric and α₁β mRNA in HEK 293 cells generated glycine receptors that showed glycine-activated inward current with amplitudes of −1.0±0.5 nA in α₁ and 1.3±0.9 nA in α₁β receptors following saturating (1000 μM) concentrations of the natural agonist. Successful co-expression of the β subunit was verified with picrotoxin 1000 μM co-applied with 1000 μM glycine after each experiment. In this experimental setting, picrotoxin 1000 μM blocked α₁ homomeric receptors by 55±0.05% while α₁β receptors were hardly affected by picrotoxin (19±0.05% block). When α and β cDNAs were used at a 1:10 ratio for co-transfection, successful co-expression of the β-subunit verified with picrotoxin was 100%. The current transient showed a fast increase, followed by a monophasic decay. The time constant of desensitization was 958±250 ms in α₁ homomeric and 1026±212 ms in α₁β receptors. The respective steady-state current that did not desensitize in the presence of 1000 μM glycine was at 86±6% and 84±8% of the peak current amplitude.

When applied without glycine, only 2,6 dimethylphenol and 3,5 dimethylphenol directly activated receptor-mediated inward currents in a concentration-dependent manner. Currents reached 30±12% (α₁, n=3) and 38±5% (α₁β, n=3) and 32±6% (α₁, n=3) and 33±10% (α₁β, n=3) of the maximum glycine (1000 μM) response in the presence of high concentrations (3000 μM) of either 3,5 dimethylphenol or 2,6 dimethylphenol, respectively. The estimates for half-maximum concentrations (EC₅₀) were 1468±208 and 1466±83 μM for 3,5 dimethylphenol and 1410±101 and 1549±164 μM for 2,6 dimethylphenol in α₁ and α₁β receptors, respectively.

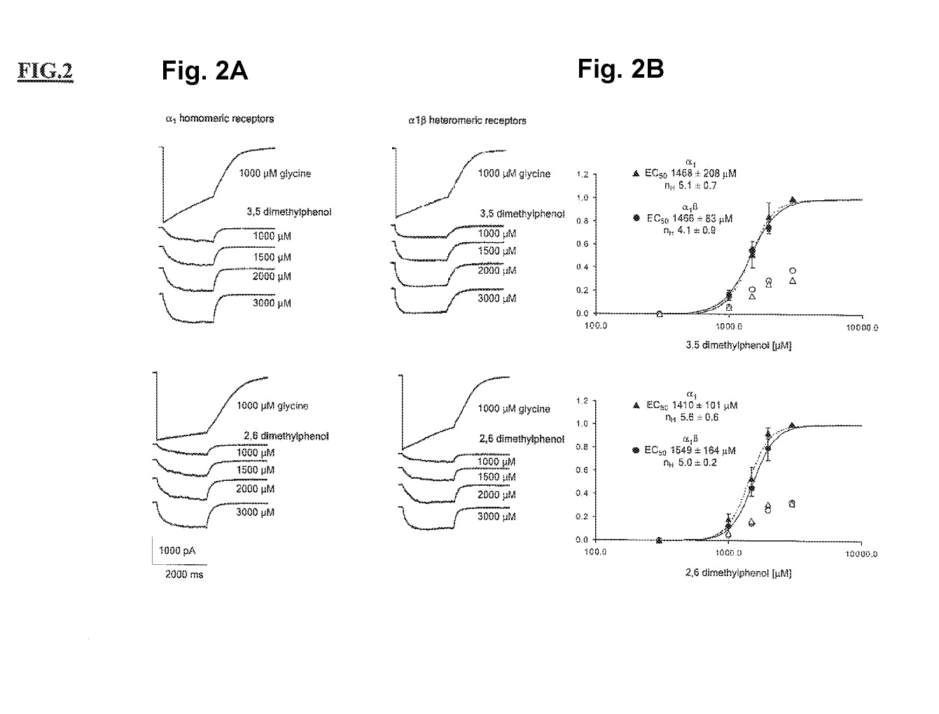

As illustrated by the tracings in FIG. 2, currents induced by both compounds did not desensitize during the 2 s application.

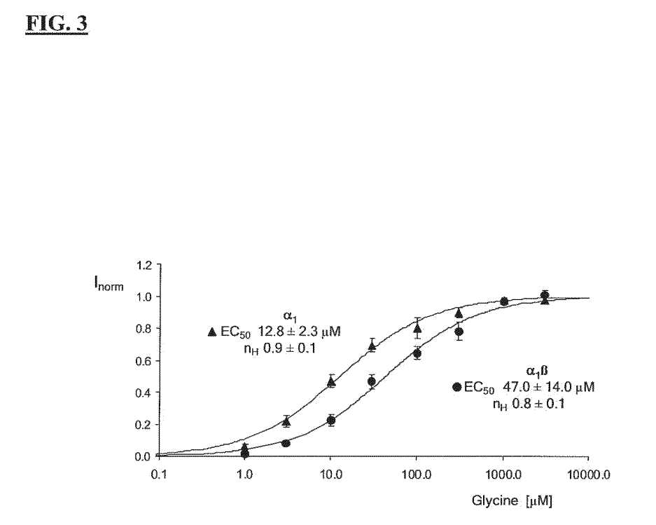

Dose-response curves for glycine at α₁ and α₁β receptors are shown in FIG. 3. The EC₅₀ for glycine was 12.8±2.3 μM at α₁ and 47.0±14.0 μM at α₁β receptors, respectively. Glycine 10 μM evoked a current response of 21±7% (n=34) in α₁β and 46±5% (n=24) of the response to 1000 μM glycine in α₁ receptors, this difference in glycine sensitivity was significant (p<0.001). Less than 10% of the current response to 10 μM glycine desensitized as long as glycine was present.

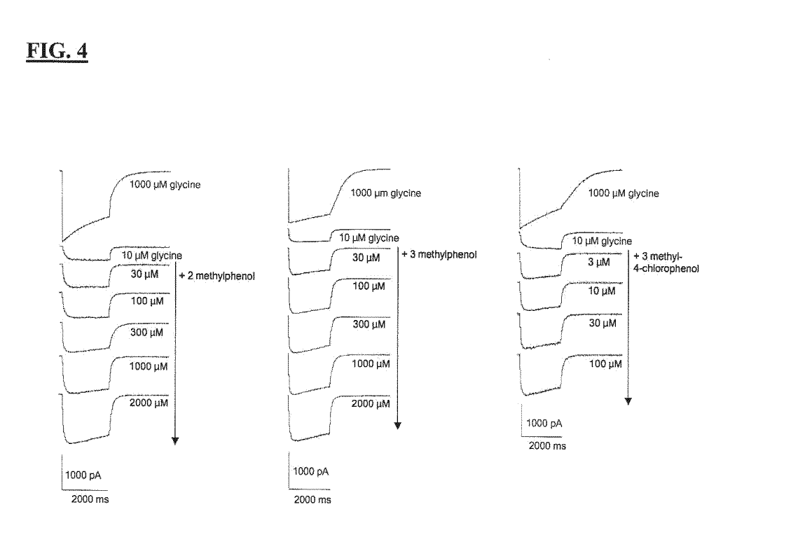

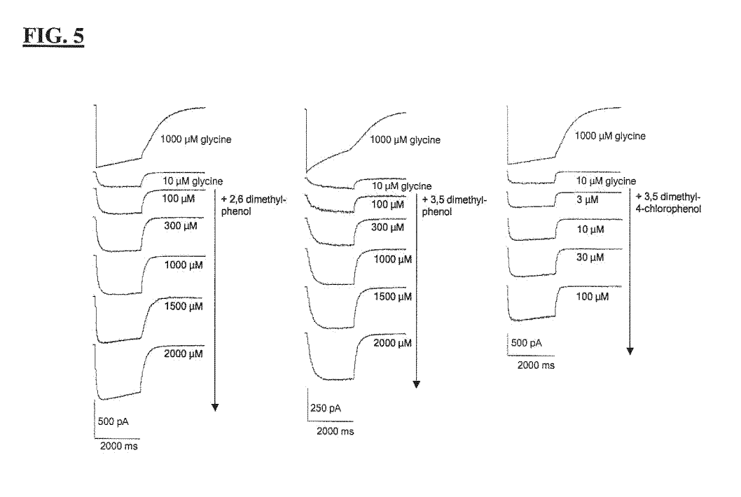

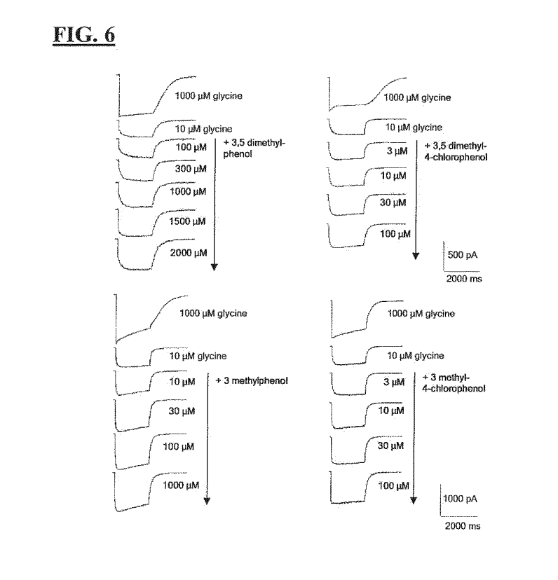

All phenol derivatives investigated potentiated the current response to glycine 10 μM in both α₁ and α₁β receptors, FIGS. 4 and 5 show representative current traces obtained with α₁β receptors, FIG. 6 shows current traces obtained with α₁ homomeric receptors.

No significant differences between the compounds were detected with respect to the degree of maximum potentiation. Only the potentiating effect seen with 3 methylphenol was higher than with 2 methylphenol (p=0.04), which, however, might be a consequence of the lower response to glycine 10 μM in the experiments with 3 methylphenol with respect to the experiments with 2 methylphenol.

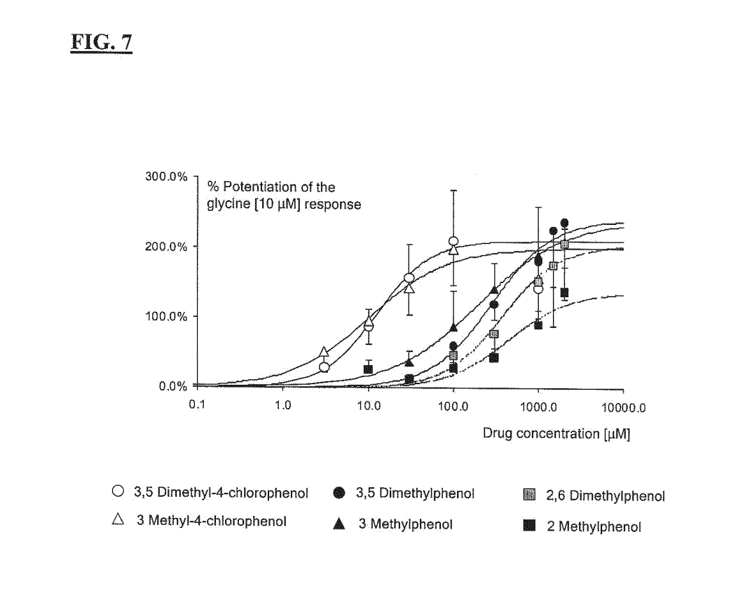

The halogenated compounds 3,5 dimethyl-4-chlorophenol and 3 methyl-4-chlorophenol achieved half-maximum potentiating effects at more than 20-fold lower concentrations compared with their non-halogenated analogues, this difference was surprising to the inventors and was statistically significant (p<0.0001). The estimates for the EC₅₀ values for the compounds with the methyl groups in the meso position (3 methylphenol and 3,5 dimethylphenol) in α₁β receptors were not significantly different from the EC₅₀ values for their ortho-methylated structural analogues (2 methylphenol and 2,6 dimethylphenol). The noon-halogenated bimethylated compounds were not significantly more potent than their structural analogues with only one methyl group in α₁β receptors. The concentration-dependence of current potentiation in α₁β receptors derived from 5-6 experiments for each compound is depicted in FIG. 7.

The EC₅₀ values and Hill-coefficients (±SD) derived from fits of the Hill equation to the normalized response in α₁ and α₁β receptors are depicted in Table 1.

TABLE 1
EC₅₀ values and Hill coefficients (+s.d.) derived
from fits of the Hill equation to the normalised coactivating
response (with respect to the effect of the highest concentration
tested) in α₁ and α₁β receptors

| | α₁ homomer | | α₁β heteromer | |
|---|---|---|---|---|
| | EC₅₀ (μM) | n_{H} | EC₅₀ (μM) | n_{H} |
| 3 methyl-4-chlorophenol | 8 ± 5 | 1.1 ± 0.4 | 4 ± 1* | 1.2 ± 0.3 |
| 3 methylphenol | 59 ± 19 | 0.9 ± 0.3 | 222 ± 45 | 1.0 ± 0.1 |
| 3,5 dimethyl-4-chloro- | 13 ± 4 | 1.4 ± 2.9 | 11 ± 2* | 1.3 ± 0.1 |
| phenol | | | | |
| 3,5 dimethylphenol | 254 ± 139 | 1.7 ± 0.9 | 308 ± 46 | 1.3 ± 0.2 |
| 2 methylphenol | 70 ± 29 | 0.7 ± 0.1 | 448 ± 89 | 1.2 ± 0.2 |
| 2,6 dimethylphenol | 226 ± 104 | 1.5 ± 0.4 | 373 ± 51 | 1.2 ± 0.1 |

In Table 1: the halogenated compounds 3 methyl-4-chlorophenol and 3,5 dimethyl-4-chlorophenol were significantly more potent than their nonhalogenated structural analogues (bold and *, P<0.0001). No significant differences were detected between compounds with one vs two methyl groups or between ortho- vs meso-methylated compounds in α₁β receptors.

As a consequence of the higher glycine sensitivity in α₁ homomeric receptors, a maximum co-activating response (with respect to the effect of 1000 μM glycine) might occasionally be observed at low drug concentrations leading to an underestimation of the EC₅₀ values derived from Hill-fits in α₁ homomeric receptors—thus, the parameters given for the α₁ homomeric receptors should not be used for potency determinations. However, as revealed by the current traces in FIGS. 4, 5 and 6 and by the values given in Table 1, all phenol derivatives co-activate currents via α₁ homomeric receptors in a similar concentration range compared to α₁β receptors, thus, the expression of the β-subunit is not required for the co-activating effects.

The halogenated compounds 3,5 dimethyl-4-chlorophenol and 3 methyl-4-chlorophenol in concentrations larger than 300 and 600 μM, respectively, produced a reduction in the peak current amplitude when co-applied with 1000 μM glycine along with a large response rebound when co-application was terminated. The current decay was accelerated during co-application of the respective compound and glycine (1000 μM). A total of 3 experiments was performed for each compound to substantiate this effect. FIG. 8 shows representative current traces.

### 1.3 Discussion

These data shows that substituted phenol derivatives that carry a chloride in the para position to the phenolic hydroxyl group co-activate glycine receptors at low concentrations and thus, may offer a potential for therapy of spasticity, muscle relaxation, and pain relief. At much higher concentrations, only the bi-methylated and non-halogenated compounds directly activated the glycine receptor in the absence of the natural agonist. These results show that direct activation and co-activation of glycine receptors by phenol derivatives require distinct structural features. The presence of the β subunit is neither required for positive modulation nor for direct activation of glycine receptors by phenol derivatives.

GABA_{A} and glycine receptors are the main receptors for inhibitory neurotransmission in the mammalian central nervous system. GABA_{A} is the most important neurotransmitter in the brain and glycine plays a major role in the spinal cord and lower brain stem. While GABA_{A} receptors have been identified as a common target site for structurally diverse sedative-anaesthetic and anxiolytic drugs, clinically applicable compounds that specifically target glycine receptors have yet to be identified. Glycine receptors have been suggested as potential candidates for therapeutics that mediate anti-nociceptive and muscle relaxant effects.

All phenol derivatives investigated in this study were capable of positively modulating glycine receptor function to a certain extent. Apparently, one important structural feature that determines the potency of a phenol derivative to co-activate glycine receptors is halogenation in the para position to the phenolic hydroxyl group. Insertion of a second symmetrical methyl group did not further increase the potency of the single-methylated compound, and the position of the methyl group with respect to the phenolic hydroxyl group had no influence on the co-activating potency. At higher concentrations (>300 μM), the co-activating effect of the halogenated compounds was overridden by inhibitory effects revealed by a reduction in the peak current amplitude during co-application with 1000 μM glycine. The large response rebound when co-application was stopped simultaneously is consistent with the assumption of open channel block as underlying mechanism—a phenomenon that has previously been described for the modulation of glycine receptors by high concentrations of propofol as well as for the modulation of GABA_{A} receptors by inhalational agents. However, further studies should target voltage-dependence of these effects in order to substantiate the hypothesis of open-channel block. Alternatively, the reduction in peak current amplitude along with the acceleration of the current decay during co-application with 1000 μM glycine might be explained by an allosteric mechanism of inhibition with high concentrations of halogenated phenol derivatives stabilizing the desensitized conformation of the receptor, analogous to a mechanism of block assumed for picrotoxin on ligand-gated chloride channels. None of the halogenated compounds directly activated the receptor in the absence of the natural agonist.

The structural features that determine the potency of a phenol derivative to activate or co-activate Cl⁻ inward currents via glycine receptors show similarities as well as differences with respect to the requirements that have previously been reported for activation of GABA-ergic receptors or sodium channel blocking effects.

Qualitatively, the structure-activity relationship for co-activation of glycine receptors by phenol derivatives shows parallels with the structure-activity relationship to block voltage-operated sodium channels. In both cases, potency is strongly increased by the chloride in para position to the phenolic hydroxyl. Quantitatively, the half-maximum concentrations for glycine receptor co-activation in this study were about 10-fold (3,5 dimethyl-4-chlorophenol) and 100-fold (3 methyl-4-chlorophenol) lower than the concentrations required for half-maximum blockade of sodium channels by these compounds. While insertion of a chloride in para position led to a parallel increase in the potency to co-activate glycine receptors and to block of voltage-operated sodium channels, this is not the case for the insertion of a 2^{nd} methyl group. In contrast to the effect at glycine receptors, the potency for sodium channel blockade was increased when a second methyl group was attached in the meso position. As for the halogenated compound with one single methyl group in the meso position (3 methyl-4-chlorophenol), there is only little overlap in the concentration range where glycine receptor co-activation was observed in this study and the concentration range where sodium channel blockade was reported. For comparison, half-maximum effect at glycine receptors was achieved with 4 μM 3-methyl-4-chlorophenol, whereas half-maximum block of sodium channels in the resting state required 400 μM 3-methyl-4-chlorophenol. In the case of the non-halogenated phenol derivatives with two methyl groups, co-activation of glycine receptors was detected in the same concentration range as sodium channel blockade. In this study, a half-maximum co-activating effect was observed with 370 μM 2,6 dimethylphenol compared to 187 μM for half-maximum blockade of voltage-operated neuronal sodium channels at a membrane potential close to the physiological resting potential. At much higher concentrations (>1000 μM), the bi-methylated compounds directly activated the glycine receptor in the absence of the natural agonist. If these results can be generalized, halogenated phenol derivatives should show glycine receptor co-activation at low concentrations, with increasing concentrations blockade of voltage-operated sodium channels and open channel block of glycine receptors. Non-halogenated bi-methylated phenol derivatives should both co-activate glycine receptors and block sodium channels at intermediate concentrations and should directly activate glycine receptors at high concentrations. Halogenated phenol derivatives with one single methyl group would be expected to be sodium channel blockers, while having facilitating effects at glycine receptors at concentrations where sodium channel blockade would still be small.

In contrast to the glycinergic effects seen in this study, GABA-ergic effects were hardly affected by substitution in the para position. GABA-ergic activity of phenolic compounds has been linked to the size and shape of alkyl groups in position 2 and 6 of the aromatic ring relative to the phenolic hydroxyl group, however, the effect of 3,5 di-alkyl-substitution has never been tested systematically. Direct activation of GABA_{A} receptors was seen in the single methylated compound only when the methyl group was in the ortho position. Our study shows that, as far as direct activation of glycine receptors is concerned, at least two methyl groups are required for a detectable effect which is independent from their position with respect to the phenolic hydroxyl group.

In conclusion, these data indicate that phenol derivatives according to the invention, which are preferably selective for glycine receptors rather than voltage-operated sodium channels or GABA_{A} receptors, show a desirable pattern of anti-nociceptive, muscle relaxant and local anaesthetic/anti-convulsant effects in the model use. A skilled person will recognise that this illustrates that compounds according to the invention will be useful in the control of a number of painful conditions as discussed herein.

### 1.4 Conclusions

1. Phenol derivatives constitute a family of neuroactive compounds. The aim of this study was to identify structural features that determine their modulatory effects at glycine receptors.

2. The inventors investigated the effects of four methylated phenol derivatives and two halogenated analogues on chloride inward currents via rat α₁ and α₁β glycine receptors, heterologously expressed in HEK 293.

3. All compounds potentiated the effect of a sub-maximal glycine concentration in both α₁ homomeric and α₁β glycine receptors. While the degree of maximum potentiation of the glycine 10 μM effect in α₁β receptors was not different between the compounds, the halogenated compounds achieved half-maximum potentiating effects in the low μM range—at more than 20-fold lower concentrations compared with their non-halogenated analogues (p<0.0001). The co-activating effect was overridden by inhibitory effects at concentration>300 μM in the halogenated compounds.

4. Only the bi-methylated compounds 2,6- and 3,5-dimethylphenol (at concentrations>1000) μM directly activated both α1 and α1β receptors up to 30% of the maximum response evoked by 1000 μM glycine.

These results show that halogenation in the para position is a crucial structural feature for the potency of a phenolic compound to positively modulate glycine receptor function while direct activation is only seen with high concentrations of compounds that carry at least 2 methyl groups. The presence of the β-subunit is not required for both effects.

### Example 2

The Experimental protocols employed in Example 1 were repeated to investigate the efficacy of a number of further halogenated propofol derivatives on glycine receptor activation (and therefore pain control).

The inventors established that compound according to general formula I with at least two of R₁, R₂, R₄ and R₅ comprising alkyl groups of between 2 and 13 carbon atoms were particularly effective for modulating glycine receptors. Such compounds are particularly preferred compounds for use according to the invention and exhibited analgesic properties that were even better than compounds according to general formula I with methyl groups at R₁, R₂, R₄ and/or R₅ (see above).

Illustrative data for preferred compounds 4-chloropropofol, 4-bromopropofol and 4-iodopropofol is set out below.

2.1: 4-chloropropofol

The experiments conducted in Example 1 were repeated using 4-chloropropofol (4-chloro-2,6 di-isopropylphenol).

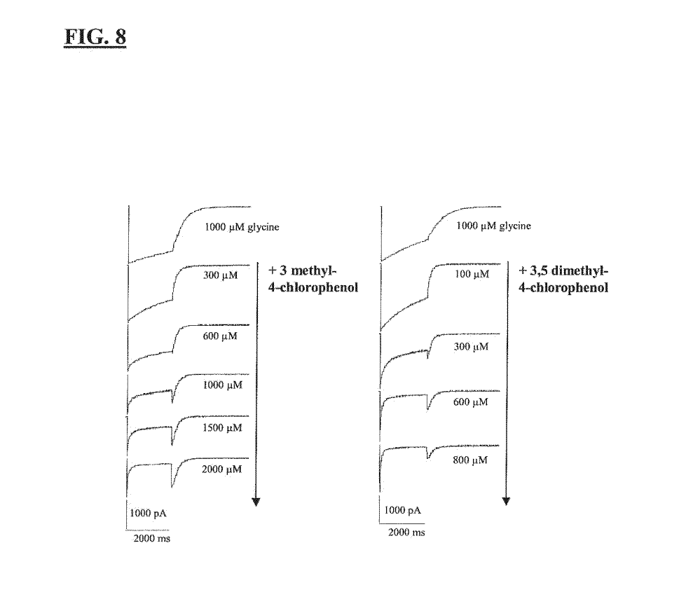

FIG. 9 illustrates representative current traces showing co-activation of the current response to 10 μM glycine when 4-chloropropofol was co-applied with 10 μM glycine (3rd, 4th, 5th and 6th current trace from top). The first trace shows current elicited by a supramaximal glycine concentration (1000 μM) a-subunits of glycine receptors from the rat were coexpressed with human β subunits in HEK293 cells. Small cells were studied in the whole-cell mode using an ultra-fast application device.

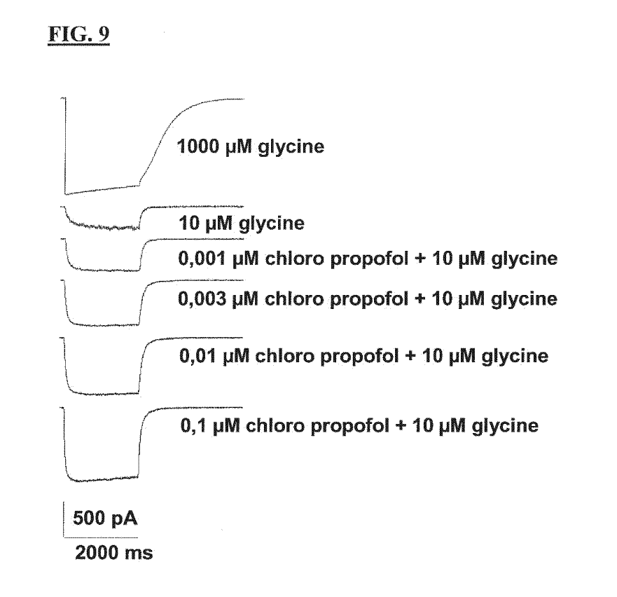

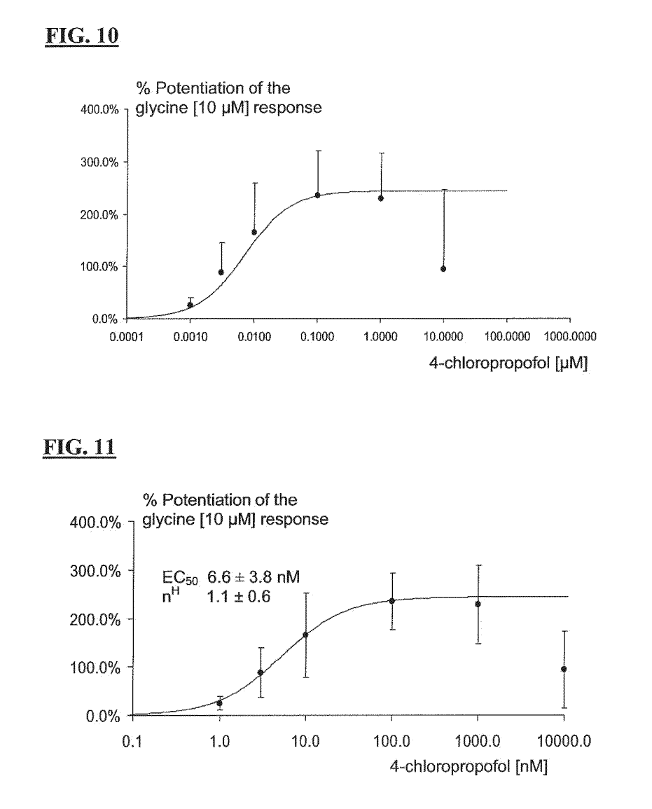

FIG. 10 illustrates concentration-dependence of the coactivation of the current response to 10 μM glycine by 4-chloropropofol (mean/SD, n=6). The results presented in FIG. 10 were corroborated by repeating the tests carried out to provide the data shown in FIG. 10 to provide the data shown in FIG. 11. Thus, FIG. 11 also illustrates concentration-dependence of the coactivation of the current response to 10 μM glycine by 4-chloropropofol (mean/SD, n=5). Note from FIGS. 10 and 11 that the co-activating effect is observed in the concentration range between 1 and 100 nM. The co-activating effect is overridden by inhibitory effects at higher concentrations (≧1000 μM). The decline in the peak current amplitude at concentrations above 10 μM is probably due to open channel block at higher concentrations, a phenomenon that has been described for propofol. a-subunits of glycine receptors from the rat were coexpressed with human β-subunits in HEK293 cells. Small cells were studied in the whole-cell mode using an ultra-fast application device.

FIG. 12 represents current traces elicited by 4-chloropropofol in the absence of the natural agonist glycine (2nd, 3rd and 4th trace from top) with respect to the current elicited by a supramaximal glycine concentration (1000 μM), top. a-subunits of glycine receptors from the rat were coexpressed with human β-subunits in HEK293 cells. Small cells were studied in the whole-cell mode using an ultra-fast application device.

These data have also been confirmed in experiments using Oocytes instead of HEK293 cells as expression system.

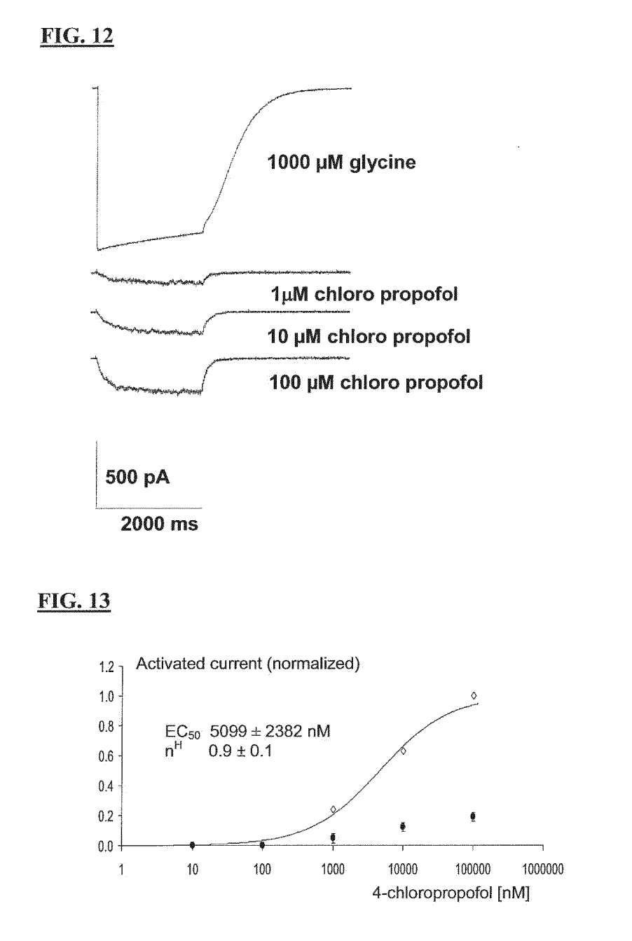

FIG. 13 shows the current directly activated (mean±SD, n=4) by 4-chloropropofol normalized to its own maximum response (empty symbols) or the maximum response elicited by 1000 μM glycine (filled symbols). The solid line is a Hill fit to the data with the indicated parameters. Note that the direct activating effect is observed at 1000-fold higher concentrations than the co-activating effect.

2.2: 4-bromopropofol

The experiments conducted in Example 1 were repeated using 4-bromopropofol (4-bromo-2,6 di-isopropylphenol).

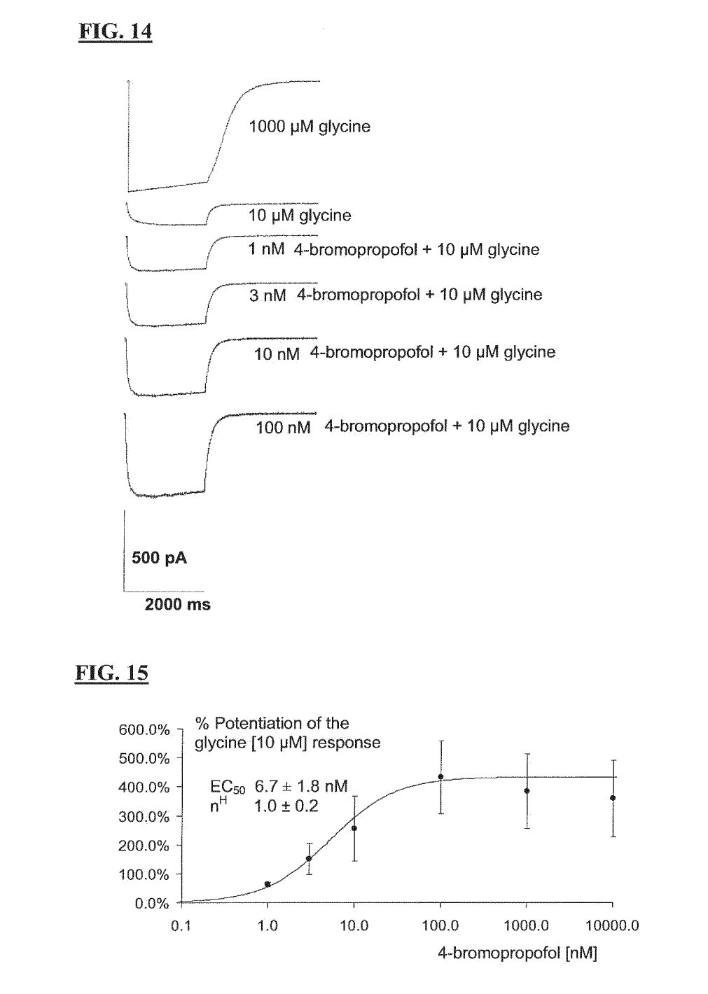

FIG. 14 illustrates representative current traces showing co-activation of the current response to 10 μM glycine when 4-bromopropofol was co-applied with 10 μM glycine (3rd, 4th, 5th and 6th current trace from top). The first trace shows current elicited by a supramaximal glycine concentration (1000 μM) a-subunits of glycine receptors from the rat were coexpressed with human β subunits in HEK293 cells. Small cells were studied in the whole-cell mode using an ultra-fast application device.

FIG. 15 illustrates concentration-dependence of the coactivation of the current response to 10 μM glycine by 4-bromopropofol (mean/SD, n=5). Note from FIG. 15 that the co-activating effect is observed in the concentration range between 1 and 100 nM. The co-activating effect is overridden by inhibitory effects at higher concentrations (>1000 μM).

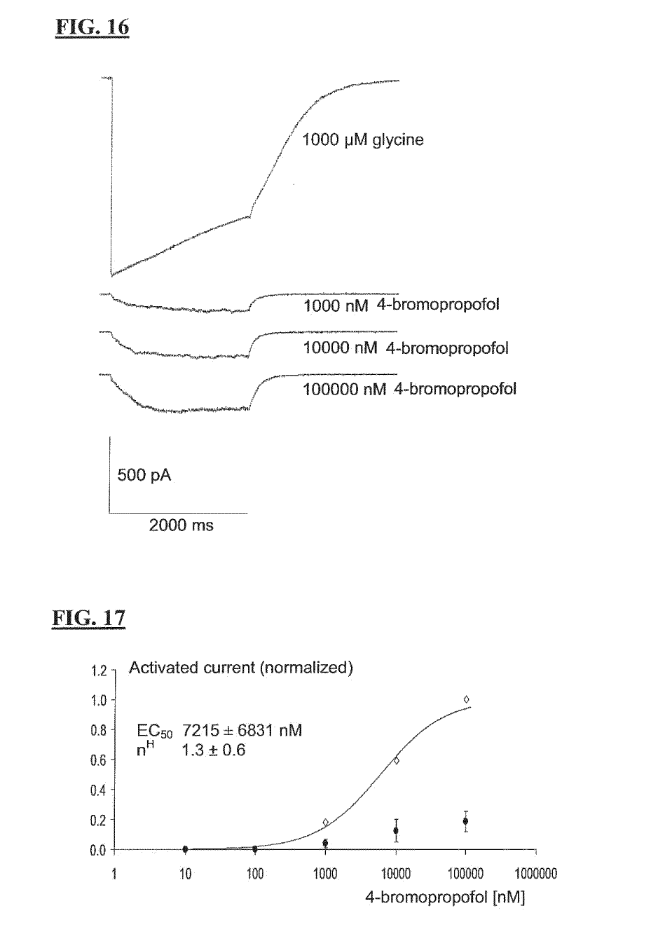

FIG. 16 represents current traces elicited by 4-bromopropofol in the absence of the natural agonist glycine (2nd, 3rd and 4th trace from top) with respect to the current elicited by a supramaximal glycine concentration (1000 μM), top. a-subunits of glycine receptors from the rat were coexpressed with human β-subunits in HEK293 cells. Small cells were studied in the whole-cell mode using an ultra-fast application device.

FIG. 17 shows the current directly activated (mean±SD, n=4) by 4-bromopropofol normalized to its own maximum response (empty symbols) or the maximum response elicited by 1000 μM glycine (filled symbols). The solid line is a Hill fit to the data with the indicated parameters. Note that the direct activating effect is observed at 1000-fold higher concentrations than the co-activating effect.

2.3: 4-iodopropofol

The experiments conducted in Example 1 were repeated using 4-iodopropofol (4-iodo-2,6 di-isopropylphenol).

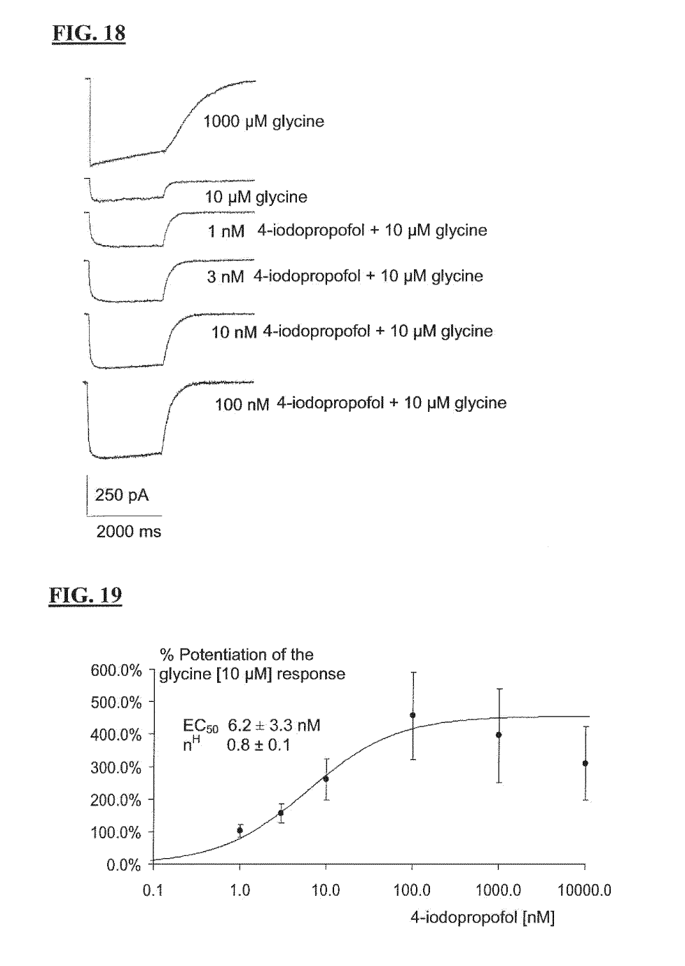

FIG. 18 illustrates representative current traces showing co-activation of the current response to 10 μM glycine when 4-iodopropofol was co-applied with 10 μM glycine (3rd, 4th, 5th and 6th current trace from top). The first trace shows current elicited by a supramaximal glycine concentration (1000 μM) a-subunits of glycine receptors from the rat were coexpressed with human β subunits in HEK293 cells. Small cells were studied in the whole-cell mode using an ultra-fast application device.

FIG. 19 illustrates concentration-dependence of the coactivation of the current response to 10 μM glycine by 4-iodopropofol (mean/SD, n=4). Note from FIG. 19 that the co-activating effect is observed in the concentration range between 1 and 100 nM. The co-activating effect is overridden by inhibitory effects at higher concentrations (>1000 μM).

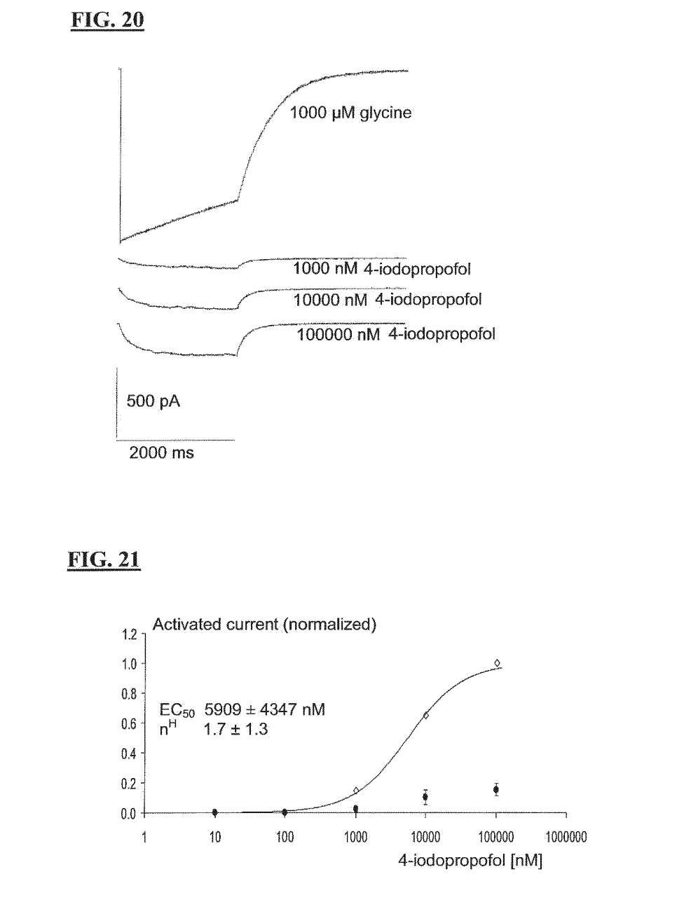

FIG. 20 represents current traces elicited by 4-iodopropofol in the absence of the natural agonist glycine (2nd, 3rd and 4th trace from top) with respect to the current elicited by a supramaximal glycine concentration (1000 μM), top. a-subunits of glycine receptors from the rat were coexpressed with human β-subunits in HEK293 cells. Small cells were studied in the whole-cell mode using an ultra-fast application device.

FIG. 21 shows the current directly activated (mean±SD, n=4) by 4-iodopropofol normalized to its own maximum response (empty symbols) or the maximum response elicited by 1000 μM glycine (filled symbols). The solid line is a Hill fit to the data with the indicated parameters. Note that the direct activating effect is observed at 1000-fold higher concentrations than the co-activating effect.

FIGS. 12, 13, 16, 17, 20 and 21 provide experimental data for the effect of the 4-chloro-, 4-bromo- and 4-iodo-derivatives of 2,6-di-isopropylphenol when provided in the absence of the natural transmitter glycine. The three compounds exhibited EC₅₀ values in the single-digit micromolar range, i.e. three orders of magnitude higher than in the presence of glycine (see FIGS. 11, 15 and 19). While the inventors do not wish to be bound by any specific theory, the results of these tests suggest that the three 4-halo-propofol derivatives do not exert a direct effect, but rather, that they modulate the effect of the natural transmitter on gating of glycine receptors.

### 2.4 Further Experimental Data

Further experiments were conducted with 4-chloropropofol that confirmed the efficacy of compounds according to the invention. These included:

(a) Preliminary experiments 100 nM 4-chloropropofol greatly enhanced the glycine (EC10)-evoked current mediated by recombinant glycine alpha1 receptors expressed in Xenopus oocytes. These data support the data about the effects of 4-chloropropofol on recombinant receptors expressed in human cell lines (HEK 293) but utilising a completely different expression system.
(b) In a further series of preliminary experiments using a rat in vitro spinal cord preparation 100 nM-1 μM 4-chloropropofol produced a prolongation of the sIPSCs (mediated by synaptic glycine receptors) and an increase in a tonic conductance (mediated by extrasynaptic glycine receptors). Importantly these data suggest that the potent effects of 4-chloropropofol reported for recombinant receptors expressed in human cell lines as well as in Xenopus oocytes, are reproduced for native glycine receptors in a neuronal setting.

### Example 3

### 3.1 Introduction

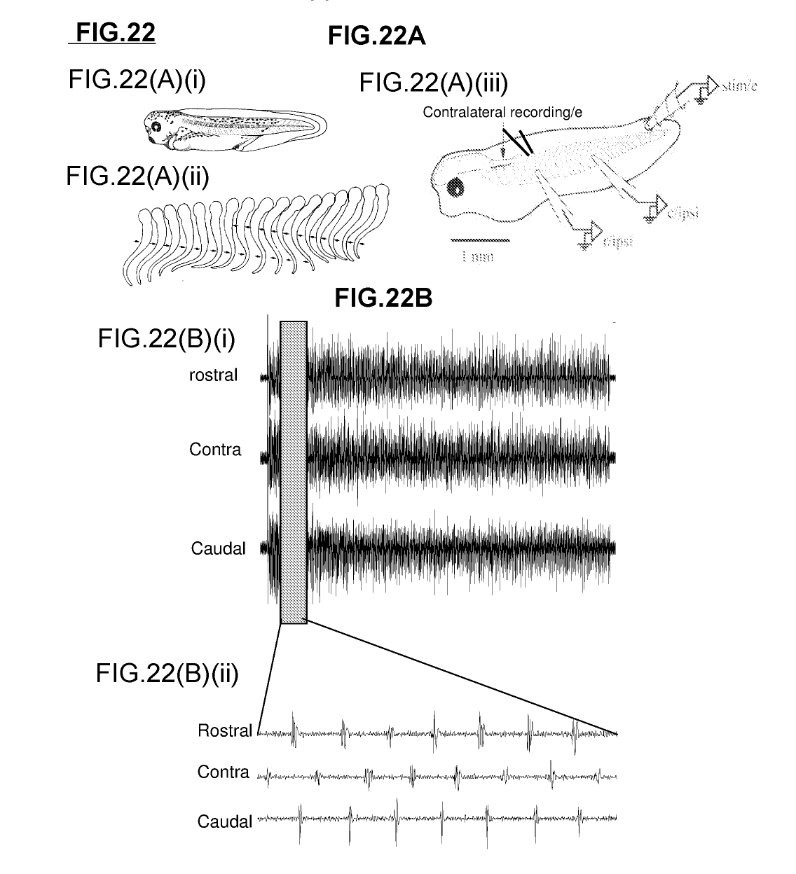

The rhythmic swimming behaviour of Xenopus laevis frog embryos (FIG. 22A) is recognised as a powerful model system for exploring spinal neural networks controlling locomotion. The intensity, frequency and duration of swimming episodes are regulated by two inhibitory pathways: a descending brainstem GABA pathway that turns off swimming and a spinal glycinergic pathway that controls the cycle periods attained during swimming. Potentiation of the GABA pathway reduces the duration of swimming episodes while potentiation of the glycinergic pathway (e.g. by noradrenaline or nitric oxide) increased cycle periods and hence slows swimming frequency.

The inventors realised that this model could be exploited to assess the relative efficacy of compounds to modulate either the GABA receptor or glycine receptors. Accordingly they were able to exploit the assay described below as a method of screening for compounds that are effective for modulating pain according to the invention.

The general anaesthetics etomidate and propofol were shown to exert an inhibitory effect upon swimming activity in Xenopus embryos by potentiating GABAergic synaptic pathways within the CNS. Propofol was also shown at higher concentrations (40 μM) to mediate effects via actions at the postsynaptic glycine receptor. This demonstrated that propofol is a potent allosteric regulator of the GABA_{A} in this system but also has actions at the glycine receptor.

In the present study Xenopus embryos were used to test the action of 4-chloropropofol (a compound according to the first aspect of the invention) upon fictive swimming in immobilized embryos. Initially 4-chloropropofol was applied in the absence of receptor antagonists to determine its anaesthetic properties compared to propofol and then the interactions of propofol with GABA and glycine receptors were assessed using bicuculline and strychnine, respectively.

### 3.2 Methods

Stage 37/8 embryos (FIG. 22Ai) were immobilized in α-bungarotoxin, secured on a Sylgard block mounted in a recording chamber and recirculated with frog ringer. The flank skin on the left and right sides of the trunk was removed to expose the inter-myotomal clefts wherein lie the ventral roots and then three glass suction electrodes were positioned at rostral and caudal levels on the left side and rostrally on the right side (FIG. 22Aiii) to record “fictive” swimming (FIG. 22B). Swimming activity was evoked by a brief 1 msec current pulse applied to the tail skin via a fourth glass suction electrode. Drugs were applied directly to the bath. Data was digitized using a CED 1401 interface, displayed using spike 2 software and analysed using Dataview software (Courtesy of W. J., Heitler, University of St. Andrews).

### 3.3 Results

### 3.3.1: 4-Chloropropofol Modulates Fictive Swimming

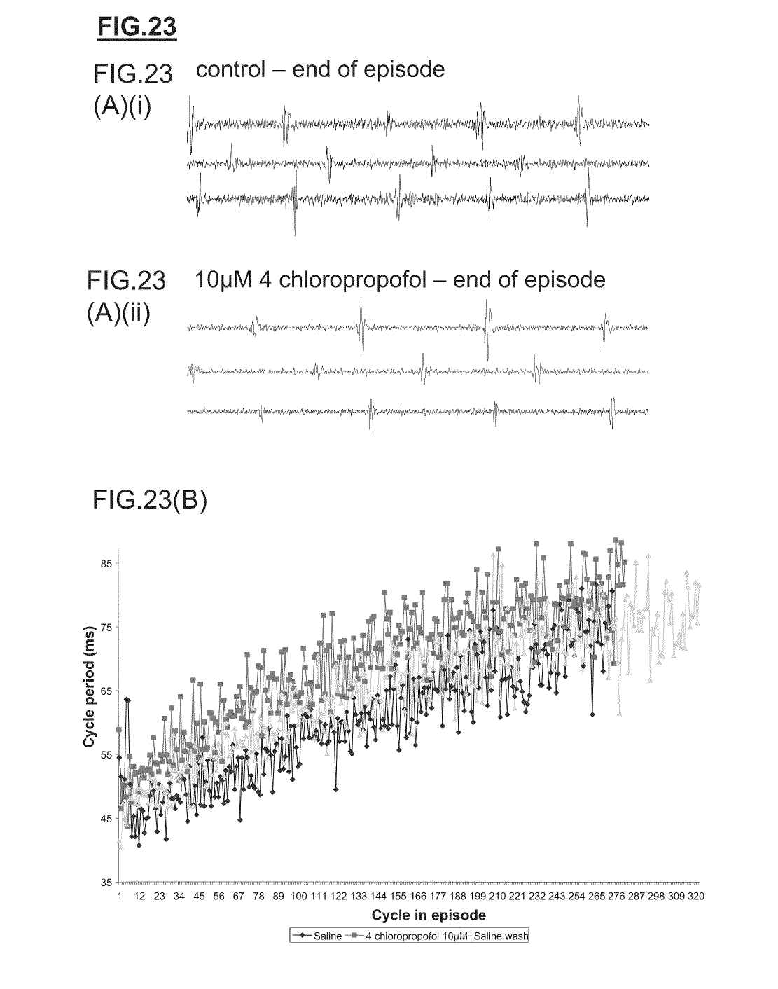

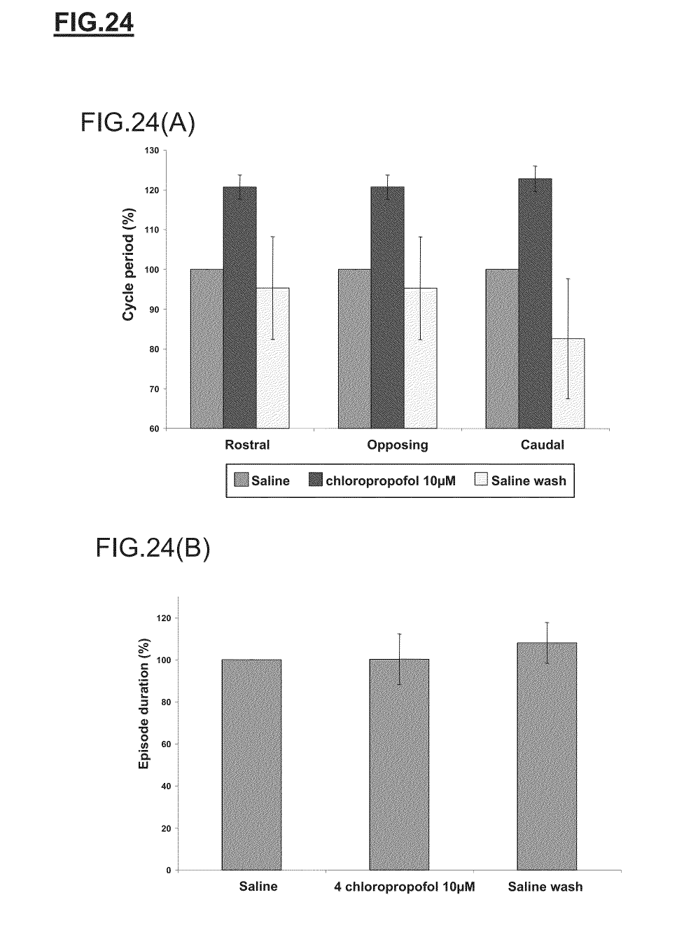

FIG. 23 shows the effect of 10 μM 4-chloropropofol on fictive swimming. 4-chloropropofol significantly increased cycle periods during swimming (FIG. 23Ai cf Aii; excerpts from end of each episode), an effect that persisted throughout each episode (FIG. 22B) and that was partially but significantly reversed by a return to control saline. In data pooled from 5 experiments, cycle periods increase by approximately 20% on average. Rostro-caudal and left-right delay also increased by 8% and 19% respectively (not illustrated), but there was no significant change in episode duration (FIG. 24B). The saline wash reversed these effects on cycle period by 25-30%, rostro-caudal delay by 12% and left-right delay by 22%. Since the cycle periods attained during swimming are regulated partly by the strength of reciprocal glycinergic inhibition these results indicate an ability of 10 μM 4-chloropropofol to potentiate glycinergic neurotransmission and therefore demonstrates that a compound according to the invention had good activity at glycine receptors. Furthermore, the lack of significant effect on episode duration suggests that 4-chloropropofol exerts no or only a weak influence upon GABAergic neurotransmission. This demonstrates that a compound according to the first aspect of the invention has good selectivity for glycine receptors, over GABA receptors in an in vivo model and is therefore useful for pain management. The selectivity of 4-chloropropofol is in contrast to the more potent effects of the anaesthetic, propofol on GABA in this system. However, in order to better establish the action of the anaesthetic, subsequent experiments utilised antagonists for firstly the GABA_{A} receptor (bicuculline methiodide) and then glycine receptor (strychnine).

### 3.3.2: 4-Chloropropofol Effects are Resistant to GABA_{A} Receptor Blockade

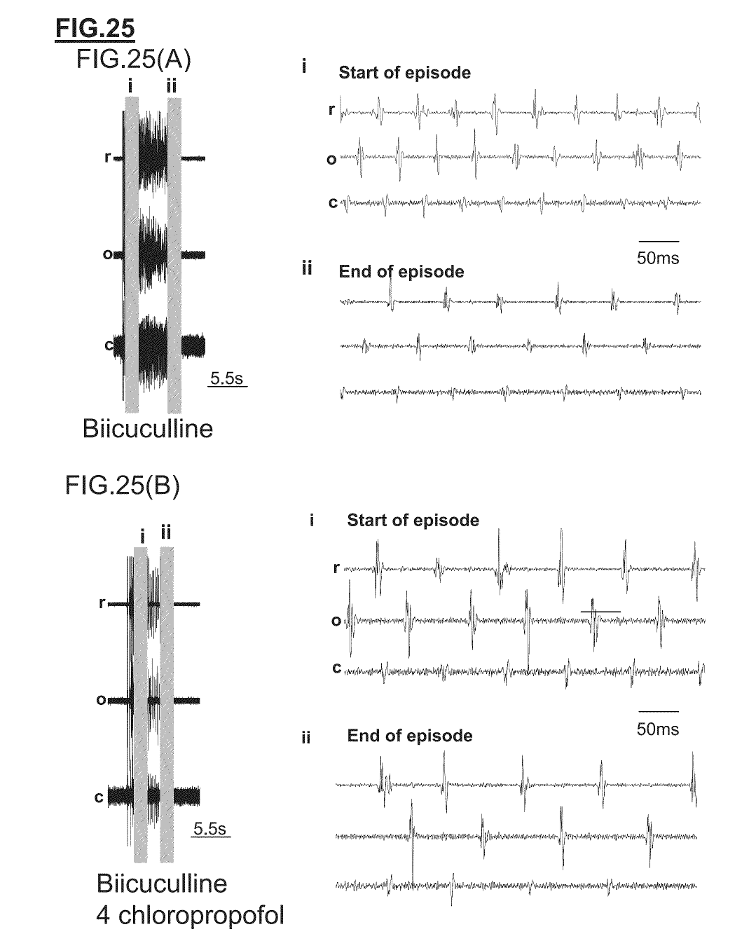

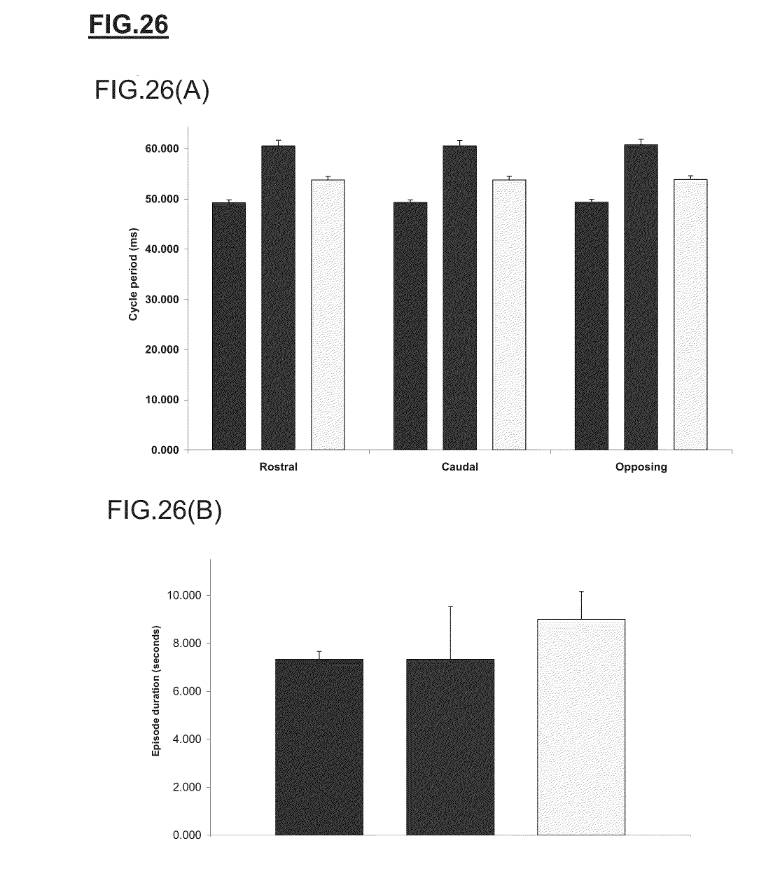

FIG. 25 shows excerpts of ventral root activity after exposure to 40 μM bicuculline methiodide to block GABA_{A} receptors (A), after the addition of 10 μM 4-chloropropofol in the presence of bicuculline (B). The effects of 4-chloropropofol on cycle periods clearly persisted after GABA_{A} receptor blockade. In this experiment, 4-chloropropofol increased episode duration (A) by 2 seconds on average and cycle periods by 8.2 ms at the start of the episode (Ai cf. Bi) and by 20.3 ms at the end of the episode (Ali cf. Bii). The bicuculline wash (not illustrated) reduced episode duration by 2 seconds and cycle period at the start of the episode by 2 ms but at the end of the episode cycle periods increased by a further 6.5 ms. Bicuculline produces a short episode of fast swimming with an average cycle period of 50.3 ms, which is increased to 61.3 ms 20 minutes after the addition of 4-chloropropofol, an effect that was reversed by the bicuculline methiodide wash to 54.5 ms.

These results demonstrate that 10 μM 4-chloropropofol mediates the majority of its actions through the potentiation of a different inhibitory receptor to the GABA_{A} receptor. In order to verify that the glycine receptor plays an important role, 1 μM Strychnine was next applied.

### 3.3.3 4-Chloropropofol Potentiates Glycinergic Pathways

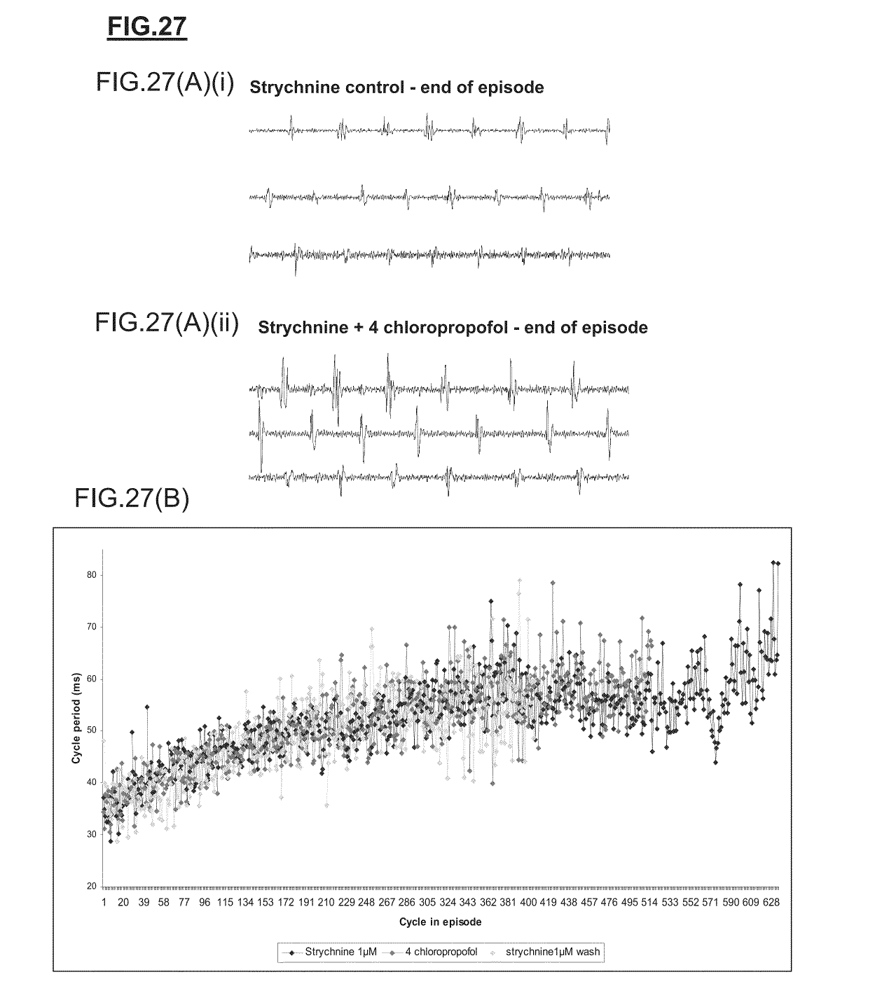

The lengthening of cycle periods by 4-chloropropofol in the presence of bicuculline suggests an action of the compound upon glycinergic transmission, for which the following experiments provide strong supporting evidence. FIG. 27 shows excerpts of ventral root activity 20 minutes after applying 1 μM strychnine (Ai) and 40 minutes after applying 10 μM 4-chloropropofol (Aii). In the presence of the compound (FIG. 27Aii), episode duration decreased by 17 seconds (A), whereas cycle periods at the start of the episode increased by an average of only 0.5 ms (Bi) and at the end of the episode decreased by an average of 3.4 ms (Bii). The strychnine wash (not illustrated) increased episode duration by 2 seconds (A) and decreased cycle periods at the start (Bi) and end of the episode (Bii) by 3 and 5.4 ms respectively. FIG. 27B illustrates the lack of effect of 4-chloropropofol on cycle periods throughout an episode of swimming. Pooled data (n=3) were used to examine the effect on the four parameters of fictive swimming when the compound is applied in the presence of 1 μM strychnine 4-chloropropofol decreased cycle periods, but not significantly, by less than 2%, which decreased a further 3% by the strychnine wash. There was little or no effect mediated by 4-chloropropofol on episode duration (B) or left-right delay (D).

### 3.3.4 Summary

At a concentration of 10 μM 4-chloropropofol potentiates inhibition mediated by glycinergic pathways, supported by its effects on the parameters of fictive swimming before and after the application of antagonists Bicuculline methiodide (40 μM) and Strychnine (1 μM). When applied alone, the compound inhibits fictive swimming through potentiating inhibitory pathways that bring about an increase in cycle periods, rostro-caudal and left-right delay with no change on episode duration. After blocking GABA_{A} receptors with 40 μM Bicuculline methiodide, 4-chloropropofol exerts a similar effect upon fictive swimming continuing to increase cycle periods and left-right delay by approximately the same percentage an effect that is reversed by the wash. Again there is little change in episode duration, but for these experiments there is a clear decrease in rostro-caudal delay. In order to determine the ability of 4-chloropropofol to potentiate glycinergic neurotransmission, 1 μM strychnine was used to block glycine receptors. There was a fractional decrease in cycle periods, no change in episode duration or left-right delay by the compound or the wash and only a small increase in rostro-caudal delay. These results provide supportive evidence for the ability of 4-chloropropofol to inhibit fictive swimming through potentiation of glycinergic pathways and to a lesser extent through the enhancement of GABAergic neurotransmission.

### 3.4 Discussion

Fictive swimming in stage 37/8 Xenopus laevis embryos is co-ordinated by a central pattern generator (CPG) primarily located within the spinal cord. This network comprises descending and commissural interneurons, and motor neurons. These neurons on opposite sides of the spinal cord fire in strict alternation thereby producing alternate muscle contractions of antagonistic sides. Excitation for swimming is produced by glutamatergic descending interneurons. The left-right alternation is brought about by the activity of glycinergic commissural interneurons mediate mid-cycle reciprocal inhibition of each CPG half centre. Fictive swimming episodes may terminate spontaneously or as a result of GABAergic mid-hindbrain reticulospinal neurons activated by pressure to the rostral cement gland which release GABA in the spinal cord to activate GABA_{A} receptors. The purpose of this study was to determine the effect of 4-chloropropofol, upon inhibitory neurotransmission within the CNS of stage 37/8 Xenopus laevis embryos.

### 3.4.1: 4-Chloropropofol Inhibits Fictive Swimming

4-chloropropofol (a compound according to the first aspect of the invention) is a derivative of the anaesthetic propofol and was expected to have a similar inhibitory effect upon the CNS of the Xenopus embryo, potentiating the GABA_{A} and glycine receptors and thereby inhibiting fictive swimming. However, as discussed in this example, 4-chloropropofol was found to have distinct effects in that it exhibited selectivity for glycine receptors (assayed as an inhibition of fictive swimming) and thereby demonstrates a utility of 4-chloropropofol as an analgesic.

The first set of experiments investigate the effects of 10 μM 4-chloropropofol; pooled data (n=5) revealed an increase in cycle period, rostro-caudal and left-right delay with no change in episode duration. After a wash in saline measurements of these parameters returned to control levels. The main effect was a decrease in swim frequency.

Propofol potentiates GABAergic neurotransmission to a greater extent than glycinergic and from this evidence a skilled person may have inferred that 4-chloropropofol would also significantly potentiate GABA_{A} transmission and thereby act as an anaesthetic. However subsequent experiments explored the effects of 10 μM P—Cl-propofol in the presence of 40 μM Bicuculline methiodide and illustrated that this surprisingly was not the case. In this experiment 4-chloropropofol gave similar results to the experiments described above comparing control saline to the compound, where cycle periods increased by a similar amount but episode duration did not alter. The effects on cycle period were reversible after returning to bicuculline alone. From this it may be concluded that 4-chloropropofol does not exert its effects on the fictive swimming frequency through allosteric binding interactions with the GABA_{A} receptor. Instead 4-chloropropofol binds to allosteric sites on the glycine receptor potentiating these inhibitory pathways and reducing fictive swimming frequency as a result.

The last set of experiments tested the hypothesis that 4-chloropropofol, and other compounds according to the invention, potentiates glycinergic neurotransmission by pre-blocking glycine receptors with 1 μM strychnine and then applying the compound. The results (n=3) revealed that the effects of 4-chloropropofol on cycle periods are occluded by strychnine; cycle periods decreased by less than 2% after addition of the 4-chloropropofol and decreased a further 1% with the strychnine wash. There was no change in episode duration or left-right delay when 4-chloropropofol was present. These results demonstrate that 10 μM 4-chloropropofol increases cycle period, rostro-caudal delay and left-right delay by enhancing glycinergic inhibition.

### 3.5 Conclusions

4-chloropropofol reduces the frequency of fictive swimming in Xenopus embryos, an effect which persists throughout each episode. However, the duration of each episode was not significantly affected. These effects are explained by a potentiation of glycinergic transmission and demonstrates in an in vivo model that compounds according to the invention are selective for glycine receptors and will therefore be useful as analgesics rather than the GABA selectivity of propofol which is useful as an anaesthetic.

Changes in the strength of glycinergic inhibition are known to exert a potent influence of swimming frequency. For example, a reduction in the inhibition (e.g. by applying strychnine) increases swimming frequency while potentiating glycinergic synapses (e.g. with noradrenaline or nitric oxide) reduces swim frequency. The effects of 4-chloropropofol cannot be accounted for by an influence on GABA neurotransmission since i) it does not significantly affect episode duration and ii) the effects persist in the presence of bicuculline at concentrations sufficient to block GABA_{A} receptors. In contrast, applications of strychnine occluded the effects of 4-chloropropofol on swimming.

These effects of 4-chloropropofol indicate a different primary site of action to propofol, which inhibited swimming via actions at the GABA_{A} receptor at low concentration (10 μM), but only affected swimming via potentiation of the glycine receptor at higher concentrations (40 μM). 4-chloropropofol appears to have a negligible effect at the GABA_{A} receptor at 1004 but exerts a powerful influence on glycine receptors at this concentration. This clearly demonstrates the surprising receptor selectivity of compounds according to the invention compared to propofol.

### Example 4

The inventors developed an in vivo pain model for further evaluation of the efficacy of the compounds according to the invention

### 4.1 Methods

Male Sprague-Dawley rats, approx 250 g, were used to test whether not the compounds had analgesic properties.

4.1.1 Pilot Study: 2 Experimental Groups (n+6/Group),
Chronic Constriction Injury (CCI)
CCI+Intrathecal Canulae (IC)+vehicle
CCI+Intrathecal Canulae (IC)+Highest of three doses

Animals are monitored for signs of toxicity with the vehicle and the compound. Animal behaviour is also assessed.

### 4.1.2 Main Experiment:

The Bennett model, which is known to the art, of neuropathic pain (loose ligation of one sciatic nerve) is employed.

Outcome measures will be thermal, von Frey or paw pressure (as described in Randell & Selitto (1957) Arch Int Pharmacodyn 4: p409-421).

5 groups (N=8 each group) will be tested comprising: Vehicle; Compound dose 1; Compound dose 2; Compound dose 3; Positive control (gabapentin 100 mg/kg/day)

Obtain values at baseline, post injury (day 7). Then commence continuous intrathecal drug treatment via miniosmotic pumps. Three further behavioural tests at days 8, 10, and 14.

### 4.2 Results

The inventors expect compounds according to the invention to have analgesic properties according to the Randell-Selitto test criteria.

1-17. (canceled)

18. A method to treat pain in a subject in need thereof comprising:
providing a compound of general formula I: 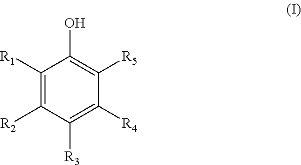
wherein R3 is a halogen;
wherein one or two of R₁, R₂, R₄ and R₅ is an alkyl comprising between 2 and 13 carbon atoms and the others are H; and
administering the compound to the subject in need thereof, so as to treat pain in the subject.

19. The method of claim 18 wherein the halogen is Chlorine or Iodine.

20. The method of claim 18 wherein two of R₁, R₂, R₄ or R₅ are an alkyl group comprising between 2 and 13 carbon atoms.

21. The method of claim 19 wherein two of R₁, R₂, R₄ or R₅ are an alkyl group comprising between 2 and 13 carbon atoms.

22. The method of claim 20 wherein the two alkyl groups are both in the ortho or meta positions.

23. The method of claim 18 wherein the compound is one of: 2,6-di-isopropyl-4-chlorophenol, 2,6-di-isopropyl-4-iodophenol, 2,6-di-isopropyl-4-fluorophenol, 2,6-di-isopropyl-4-bromophenol, 3,5-di-isopropyl-4-chlorophenol, 3,5-di-isopropyl-4-iodophenol, 3,5-di-isopropyl-4-fluorophenol and 3,5-di-isopropyl-4-bromo-phenol.

24. The method of claim 18 wherein the compound is one of: 4-bromopropofol, 4-chloropropofol and 4-iodopropofol.

25. The method of claim 18 wherein said one or two alkyl groups are isopropyl.

26. The method of claim 18 for the treatment of chronic pain.

27. The method of claim 26 wherein the chronic pain is neuropathic pain.

28. A method to treat pain in a subject in need thereof comprising:
providing a compound of general formula I: 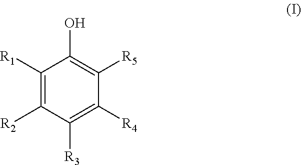
wherein R3 is a halogen;
wherein one or two of R₁, R₂, R₄ and R₅ is an alkyl comprising between 2 and 13 carbon atoms and the others are H;
wherein said one or two alkyl groups are isopropyl; and
administering the compound to the subject in need thereof, so as to treat pain in the subject.

29. The method of claim 28 wherein the halogen is Chlorine or Iodine.

30. The method of claim 28 wherein two of R₁, R₂, R₄ or R₅ are an alkyl group comprising between 2 and 13 carbon atoms.

31. The method of claim 29 wherein two of R₁, R₂, R₄ or R₅ are an alkyl group comprising between 2 and 13 carbon atoms.

32. The method of claim 30 wherein the two alkyl groups are both in the ortho or meta positions.

33. The method of claim 28 wherein the compound is one of: 2,6-di-isopropyl-4-chlorophenol, 2,6-di-isopropyl-4-iodophenol, 2,6-di-isopropyl-4-fluorophenol, 2,6-di-isopropyl-4-bromophenol, 3,5-di-isopropyl-4-chlorophenol, 3,5-di-isopropyl-4-iodophenol, 3,5-di-isopropyl-4-fluorophenol and 3,5-di-isopropyl-4-bromo-phenol.

34. The method of claim 28 wherein the compound is one of: 4-bromopropofol, 4-chloropropofol and 4-iodopropofol.

35. The method of claim 28 for the treatment of chronic pain.

36. The method of claim 35 wherein the chronic pain is neuropathic pain.
